(19) United States
(12) Patent Application Publication
(10) Pub. No.: US 2025/0086838 A1
(43) Pub. Date: Mar. 13, 2025

(54) MEDICAL SUPPORT DEVICE, ENDOSCOPE APPARATUS, MEDICAL SUPPORT METHOD, AND PROGRAM

(71) Applicant: FUJIFILM CORPORATION, Tokyo (JP)

(72) Inventor: Hiroshi UMEMOTO, Ashigarakami-gun (JP)

(21) Appl. No.: 18/822,477

(22) Filed: Sep. 3, 2024

(30) Foreign Application Priority Data
Sep. 7, 2023 (JP) 2023-145647

(51) Int. Cl. G06T 7/00; G06T 7/70; G16H 30/40; G16H 40/60

(52) U.S. Cl. CPC G06T 7/97; G06T 7/0012; G06T 7/70; G16H 30/40; G16H 40/60

(57) ABSTRACT

A medical support device includes a processor. The processor acquires position specifying information for specifying a position of a camera inserted into a body inside the body. The processor executes, based on insertion position specifying information which is the position specifying information corresponding to an insertion step frame obtained by imaging a first feature region in the body by the camera in an insertion step of inserting the camera into the body, and removal position specifying information which is the position specifying information corresponding to a removal step frame obtained by imaging a second feature region in the body by the camera in a removal step of removing the camera from the body, a first specifying process of specifying an identity between the first feature region and the second feature region.

### CROSS-REFERENCE TO RELATED APPLICATIONS

This application claims priority under 35 USC 119 from Japanese Patent Application No. 2023-145647, filed on Sep. 7, 2023, the disclosure of which is incorporated by reference herein.

### BACKGROUND

### 1. Technical Field

The present disclosure relates to a medical support device, an endoscope apparatus, a medical support method, and a program.

### 2. Related Art

JP2008-301968A discloses an endoscopic image processing device including an image acquisition unit, a lesion site detection unit, a display unit, and an image display controller. The image acquisition unit acquires an image corresponding to a subject image captured over time in an endoscope inserted into a subject. The lesion site detection unit detects a lesion site in the image each time the image is acquired. The display unit displays the image. The image display controller controls a display aspect of a plurality of images including at least a lesion site image, which is an image in which the lesion site is detected by the lesion site detection unit, among images acquired by the image acquisition unit, based on a detection result of the lesion site detection unit.

In addition, the image display controller performs control such that the display unit displays images for a predetermined period before and after a timing at which the image acquisition unit acquires the lesion site image. In addition, the image display controller performs control such that the lesion site image is displayed as a still image and an image other than the lesion site image is reproduced in reverse as a moving image, out of a series of images acquired by the image acquisition unit, which include a plurality of images from an image upon completion of insertion of the endoscope to an image upon start of the insertion and are arranged in reverse chronological order. Further, the image display controller performs control such that the lesion site image and a predetermined number of images temporally before and/or after the lesion site image are reproduced in reverse as moving images, out of the series of images acquired by the image acquisition unit, which include a plurality of images from an image upon completion of insertion of the endoscope to an image upon start of the insertion.

The endoscopic image processing device disclosed in JP2008-301968A further includes an insertion state information acquisition unit. The insertion state information acquisition unit acquires insertion state data indicating an insertion state of the endoscope inserted into the subject from an endoscope insertion state detection device, and outputs information related to the insertion state of the endoscope according to the insertion state data at a timing at which the image acquisition section acquires the lesion site image, to the display unit together with the lesion site image. Here, the information related to the insertion state of the endoscope includes at least one of an insertion length of the endoscope, an elapsed time from the insertion of the endoscope, or an insertion shape of the endoscope.

### SUMMARY

One embodiment according to the present disclosure provides a medical support device, an endoscope apparatus, a medical support method, and a program capable of specifying whether or not the same feature region is imaged by a camera in a step in which the camera advances to a far side inside a body and a step in which the camera advances to an entrance side inside the body.

A first aspect according to the present disclosure is a medical support device comprising a processor, in which the processor is configured to: acquire position specifying information for specifying a position of a camera inserted into a body inside the body; and execute, based on insertion position specifying information which is the position specifying information corresponding to an insertion step frame obtained by imaging a first feature region in the body by the camera in an insertion step of inserting the camera into the body, and removal position specifying information which is the position specifying information corresponding to a removal step frame obtained by imaging a second feature region in the body by the camera in a removal step of removing the camera from the body, a first specifying process of specifying an identity between the first feature region and the second feature region.

A second aspect according to the present disclosure is the medical support device according to the first aspect, in which the first specifying process includes a first notification process of issuing a notification that a level of the identity between the first feature region and the second feature region is equal to or higher than a first predetermined level in a case where the level of the identity between the first feature region and the second feature region is equal to or higher than the first predetermined level.

A third aspect according to the present disclosure is the medical support device according to the first or second aspect, in which the first specifying process includes a first display process of displaying a first image for specifying at least the first feature region shown in the insertion step frame and a second image for specifying at least the second feature region shown in the removal step frame on a first screen in a comparable state, in a case where a level of the identity between the first feature region and the second feature region is equal to or higher than a second predetermined level.

A fourth aspect according to the present disclosure is the medical support device according to any one of the first to third aspects, in which the processor is configured to: acquire the insertion step frame; and execute the first specifying process on a condition that a plurality of the insertion step frames, which is equal to or more than a first predetermined number of frames, are continuously acquired.

A fifth aspect according to the present disclosure is the medical support device according to any one of the first to fourth aspects, in which an insertion step identifier for identifying that the insertion step frame is a frame obtained by imaging the first feature region by the camera in the insertion step is added to the insertion step frame, and a removal step identifier for identifying that the removal step frame is a frame obtained by imaging the second feature region by the camera in the removal step is added to the removal step frame.

A sixth aspect according to the present disclosure is the medical support device according to any one of the first to fifth aspects, in which the processor is configured to execute based on pushing position specifying information which is the position specifying information corresponding to a pushing step frame obtained by imaging a third feature region in the body by the camera in a pushing step in which the camera is pushed into a far side inside the body, and pulling position specifying information which is the position specifying information corresponding to a pulling step frame obtained by imaging a fourth feature region in the body by the camera in a pulling step in which the camera is pulled from an inside of the body to a near side, a second specifying process of specifying an identity between the third feature region and the fourth feature region.

A seventh aspect according to the present disclosure is a medical support device comprising a processor, in which the processor is configured to: acquire position specifying information for specifying a position of a camera inserted into a body inside the body; and execute, based on pushing position specifying information which is the position specifying information corresponding to a pushing step frame obtained by imaging a third feature region in the body by the camera in a pushing step in which the camera is pushed into a far side inside the body, and pulling position specifying information which is the position specifying information corresponding to a pulling step frame obtained by imaging a fourth feature region in the body by the camera in a pulling step in which the camera is pulled from the far side to a near side, a second specifying process of specifying an identity between the third feature region and the fourth feature region.

An eighth aspect according to the present disclosure is the medical support device according to the sixth or seventh aspect, in which the second specifying process includes a second notification process of issuing a notification that a level of the identity between the third feature region and the fourth feature region is equal to or higher than a third predetermined level in a case where the level of the identity between the third feature region and the fourth feature region is equal to or higher than the third predetermined level.

A ninth aspect according to the present disclosure is the medical support device according to any one of the sixth to eighth aspects, in which the second specifying process includes a second display process of displaying a third image for specifying at least the third feature region shown in the pushing step frame and a fourth image for specifying at least the fourth feature region shown in the pulling step frame on a second screen in a comparable state, in a case where a level of the identity between the third feature region and the fourth feature region is equal to or higher than a fourth predetermined level.

A tenth aspect according to the present disclosure is the medical support device according to any one of the sixth to ninth aspects, in which the processor is configured to: acquire the pushing step frame; and execute the second specifying process on a condition that a plurality of the pushing step frames, which is equal to or more than a second predetermined number of frames, are continuously acquired in a case where the pushing step precedes the pulling step.

An eleventh aspect according to the present disclosure is the medical support device according to any one of the sixth to ninth aspects, in which the processor is configured to: acquire the pulling step frame; and execute the second specifying process on a condition that a plurality of the pulling step frames, which is equal to or more than a third predetermined number of frames, are continuously acquired in a case where the pulling step precedes the pushing step.

A twelfth aspect according to the present disclosure is the medical support device according to any one of the sixth to eleventh aspects, in which a pushing step identifier for identifying that the pushing step frame is a frame obtained by imaging the third feature region by the camera in the pushing step is added to the pushing step frame, and a pulling step identifier for identifying that the pulling step frame is a frame obtained by imaging the fourth feature region by the camera in the pulling step is added to the pulling step frame.

A thirteenth aspect according to the present disclosure is the medical support device according to any one of the sixth to twelfth aspects, in which the third feature region and the fourth feature region are lesions.

A fourteenth aspect according to the present disclosure is the medical support device according to any one of the first to thirteenth aspects, in which the first feature region and the second feature region are lesions.

A fifteenth aspect according to the present disclosure is the medical support device according to any one of the first to fourteenth aspects, in which the position is estimated based on a plurality of frames obtained by imaging an inside of the body by the camera.

A sixteenth aspect according to the present disclosure is the medical support device according to any one of the first to fifteenth aspects, in which the position specifying information is defined based on a movement distance of the camera inside the body.

A seventeenth aspect according to the present disclosure is an endoscope apparatus comprising: the medical support device according to any one of the first to sixteenth aspects; and the camera.

An eighteenth aspect according to the present disclosure is a medical support method comprising: acquiring position specifying information for specifying a position of a camera inserted into a body inside the body; and executing, based on insertion position specifying information which is the position specifying information corresponding to an insertion step frame obtained by imaging a first feature region in the body by the camera in an insertion step of inserting the camera into the body, and removal position specifying information which is the position specifying information corresponding to a removal step frame obtained by imaging a second feature region in the body by the camera in a removal step of removing the camera from the body, a first specifying process of specifying an identity between the first feature region and the second feature region.

A nineteenth aspect according to the present disclosure is a medical support method comprising: acquiring position specifying information for specifying a position of a camera inserted into a body inside the body; and executing, based on pushing position specifying information which is the position specifying information corresponding to a pushing step frame obtained by imaging a third feature region in the body by the camera in a pushing step in which the camera is pushed into a far side inside the body, and pulling position specifying information which is the position specifying information corresponding to a pulling step frame obtained by imaging a fourth feature region in the body by the camera in a pulling step in which the camera is pulled from the far side to a near side, a second specifying process of specifying an identity between the third feature region and the fourth feature region.

A twentieth aspect according to the present disclosure is a program for causing a computer to execute a process comprising: acquiring position specifying information for specifying a position of a camera inserted into a body inside the body; and executing, based on insertion position specifying information which is the position specifying information corresponding to an insertion step frame obtained by imaging a first feature region in the body by the camera in an insertion step of inserting the camera into the body, and removal position specifying information which is the position specifying information corresponding to a removal step frame obtained by imaging a second feature region in the body by the camera in a removal step of removing the camera from the body, a first specifying process of specifying an identity between the first feature region and the second feature region.

A twenty-first aspect according to the present disclosure is a program for causing a computer to execute a process comprising: acquiring position specifying information for specifying a position of a camera inserted into a body inside the body; and executing, based on pushing position specifying information which is the position specifying information corresponding to a pushing step frame obtained by imaging a third feature region in the body by the camera in a pushing step in which the camera is pushed into a far side inside the body, and pulling position specifying information which is the position specifying information corresponding to a pulling step frame obtained by imaging a fourth feature region in the body by the camera in a pulling step in which the camera is pulled from the far side to a near side, a second specifying process of specifying an identity between the third feature region and the fourth feature region.

### BRIEF DESCRIPTION OF THE DRAWINGS

Exemplary embodiments of the technology of the disclosure will be described in detail based on the following figures, wherein:

FIG. 1 is a conceptual diagram showing an example of an aspect in which an endoscope apparatus is used by a doctor;

FIG. 2 is a conceptual diagram showing an example of an overall configuration of the endoscope apparatus;

FIG. 3 is a block diagram showing an example of a hardware configuration of an electric system of the endoscope apparatus;

FIG. 4 is a block diagram showing an example of functions of main units of a processor included in a medical support device and an example of information stored in a storage;

FIG. 5 is a conceptual diagram showing an example of a content of a recognition process and a display content of a screen;

FIG. 6 is a conceptual diagram showing an example of an aspect in which an estimation unit executes a movement estimation process, and an example of an aspect in which a controller adds an insertion step tag or a removal step tag, a movement distance, and a recognition result to a frame;

FIG. 7 is a conceptual diagram showing an example of an aspect in which the controller stores various kinds of information in first to third storage regions of a memory;

FIG. 8 is a conceptual diagram showing an example of processing contents performed by the controller in a removal step based on a first reference movement distance stored in the first storage region, a first movement distance added to an insertion step frame stored in the second storage region, and a second movement distance added to a removal step frame stored in the third storage region;

FIG. 9 is a conceptual diagram showing an example of processing contents by the controller in a case where the removal step frame is displayed in the first display region, and the insertion step frame in which the same lesion as a lesion shown in the removal step frame is shown is displayed in the second display region, in the removal step;

FIG. 10A is a flowchart showing an example of a flow of a medical support process.

FIG. 10B is a continuation of the flowchart shown in FIG. 10A;

FIG. 11 is a flowchart showing a modification example of the medical support process;

FIG. 12 is a conceptual diagram showing an example of an aspect in which the controller performs a first notification process;

FIG. 13 is a conceptual diagram showing an example of an aspect in which a transition is made from a pushing step to a pulling step;

FIG. 14 is a conceptual diagram showing an example of an aspect in which a transition is made from the pulling step to the pushing step;

FIG. 15 is a conceptual diagram showing an example of an aspect in which the estimation unit executes the movement estimation process, and an example of an aspect in which the controller adds a pushing step tag or a pulling step tag, the movement distance, and the recognition result to the frame;

FIG. 16 is a conceptual diagram showing an example of an aspect in which the controller stores various kinds of information in fourth to sixth storage regions of the memory;

FIG. 17 is a conceptual diagram showing an example of processing contents performed by the controller based on a second reference movement distance stored in the fourth storage region, a third movement distance added to a pushing step frame stored in the fifth storage region, and a fourth movement distance added to a pulling step frame stored in the sixth storage region in a case where a transition is made from the pushing step to the pulling step in an insertion step or the removal step;

FIG. 18 is a conceptual diagram showing an example of processing contents by the controller in a case where the pulling step frame is displayed in the first display region, and a pushing step frame in which the same lesion as a lesion shown in the pulling step frame is shown is displayed in the second display region in the pulling step;

FIG. 19A is a flowchart showing an example of a flow of a first medical support process;

FIG. 19B is a continuation of the flowchart shown in FIG. 19A;

FIG. 20A is a flowchart showing an example of a flow of a second medical support process;

FIG. 20B is a continuation of the flowchart shown in FIG. 20A;

FIG. 21 is a flowchart showing a modification example of the first medical support process;

FIG. 22 is a flowchart showing a modification example of the second medical support process;

FIG. 23 is a conceptual diagram showing an example of an aspect in which the controller performs a second notification process;

FIG. 24 is a block diagram showing an example of a hardware configuration of the electric system of the endoscope apparatus equipped with an endoscope insertion shape observation device; and

FIG. 25 is a conceptual diagram showing an example of a series of pieces of processing in which a processor included in a computer gives a processing execution request to an external device via a network, the external device executes processing according to the processing execution request, and the processor included in the computer receives a processing result from the external device.

### DETAILED DESCRIPTION

Hereinafter, examples of embodiments of a medical support device, an endoscope apparatus, a medical support method, and a program according to the present disclosure will be described with reference to the accompanying drawings.

First, the wording used in the following description will be described.

CPU is an abbreviation for a “central processing unit”. GPU is an abbreviation for a “graphics processing unit”. GPGPU is an abbreviation for “general-purpose computing on graphics processing units”. APU is an abbreviation for an “accelerated processing unit”. TPU is an abbreviation for a “tensor processing unit”. RAM is an abbreviation for a “random access memory”. NVM is an abbreviation for a “non-volatile memory”. EEPROM is an abbreviation for an “electrically erasable programmable read-only memory”. ASIC is an abbreviation for an “application specific integrated circuit”. PLD is an abbreviation for a “programmable logic device”. FPGA is an abbreviation for a “field-programmable gate array”. SoC is an abbreviation for a “system-on-a-chip”. SSD is an abbreviation for a “solid state drive”. USB is an abbreviation for a “universal serial bus”. HDD is an abbreviation for a “hard disk drive”. EL is an abbreviation for “electro-luminescence”. CMOS is an abbreviation for a “complementary metal oxide semiconductor”. CCD is an abbreviation for a “charge coupled device”. AI is an abbreviation for “artificial intelligence”. BLI is an abbreviation for “blue light imaging”. LCI is an abbreviation for “linked color imaging”. I/F is an abbreviation for an “Interface”. SSL stands for a “sessile serrated lesion”. LAN is an abbreviation for a “local area network”. WAN is an abbreviation for a “wide area network”. 5G is an abbreviation for a “5th generation mobile communication system”.

In the following description, a processor with a reference (hereinafter, simply referred to as a “processor”) may be one computing device or a combination of a plurality of computing devices. In addition, the processor may be one type of a computing device or a combination of a plurality of types of computing devices. Examples of the computing device include a CPU, a GPU, a GPGPU, an APU, or a TPU.

In the following description, a memory with a reference is a memory such as a RAM in which information is temporarily stored, and is used as a work memory by the processor.

In the following description, a storage with a reference is one or a plurality of non-volatile storage devices that store various programs, various parameters, and the like. Examples of the non-volatile storage device include a flash memory, a magnetic disk, or a magnetic tape. In addition, other examples of the storage include a cloud storage.

In the following embodiment, an external I/F with a reference transmits and receives various types of information between a plurality of devices connected to each other. Examples of the external I/F include a USB interface. A communication I/F including a communication processor, an antenna, and the like may be applied to the external I/F. The communication I/F performs communication between a plurality of computers. Examples of a communication standard applied to the communication I/F include a wireless communication standard including 5G, Wi-Fi (registered trademark), or Bluetooth (registered trademark).

In the following embodiments, “A and/or B” is synonymous with “at least one of A or B”. That is, “A and/or B” means that it may be only A, only B, or a combination of A and B. In addition, in the present specification, in a case where three or more matters are associated and represented by “and/or”, the same concept as “A and/or B” is applied.

### First Embodiment

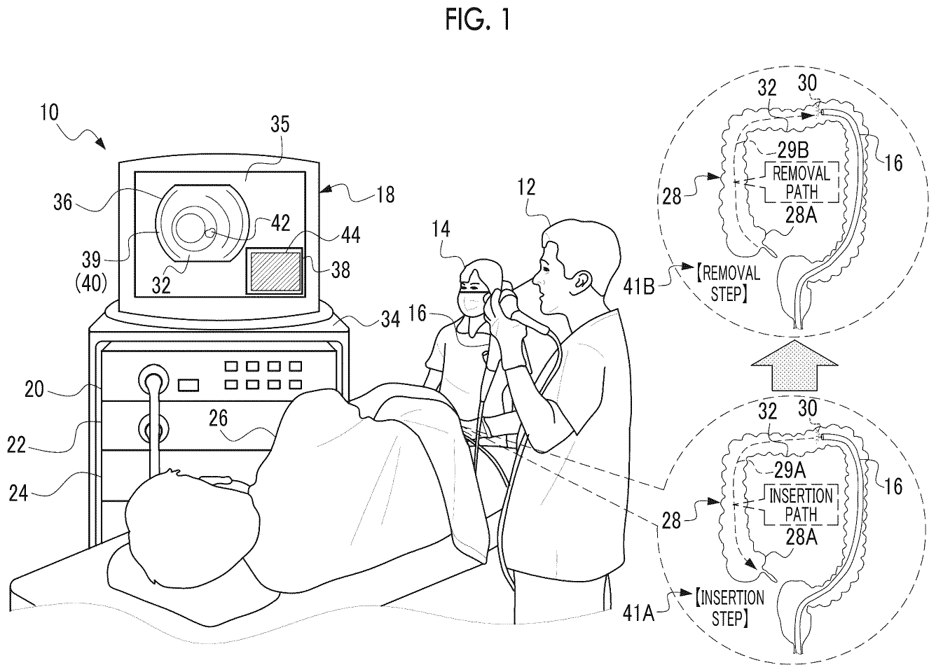

FIG. 1 is a conceptual diagram showing an example of an aspect in which an endoscope apparatus 10 is used. As shown in FIG. 1, the endoscope apparatus 10 is used by a doctor 12 in an endoscopy or the like. The endoscopy is assisted by a staff such as a nurse 14.

The endoscope apparatus 10 is connected to a communication device (not shown) in a communicable manner, and information obtained by the endoscope apparatus 10 is transmitted to the communication device. Examples of the communication device include a server, a personal computer, and/or a tablet terminal that manage various types of information such as an electronic medical record. The communication device receives the information transmitted from the endoscope apparatus 10 and executes a process using the received information (for example, a process of storing the information in an electronic medical record or the like).

The endoscope apparatus 10 comprises an endoscope 16, a display device 18, a light source device 20, a control device 22, and a medical support device 24. In the first embodiment, the endoscope apparatus 10 is an example of an “endoscope apparatus” according to the present disclosure.

The endoscope apparatus 10 is a modality for performing medical care on a large intestine 28 included in a body of a subject 26 (for example, a patient) by using the endoscope 16. In the first embodiment, the large intestine 28 is a target to be observed by the doctor 12.

The endoscope 16 is used by the doctor 12 and is inserted into the body of the subject 26. In the first embodiment, the endoscope 16 is inserted into the large intestine 28 which is a luminal organ of the subject 26.

The endoscope apparatus 10 causes the endoscope 16 inserted into the large intestine 28 of the subject 26 to image the inside of the large intestine 28 of the subject 26 and performs various medical treatments on the large intestine 28 as necessary.

The endoscope apparatus 10 acquires and outputs an image showing an aspect in the large intestine 28 by imaging the inside of the large intestine 28 of the subject 26. In the first embodiment, the endoscope apparatus 10 is an endoscope apparatus having an optical imaging function of capturing reflected light obtained by being reflected by an intestinal wall 32 of the large intestine 28 by irradiating the inside of the large intestine 28 with light 30.

Although the endoscopy of the large intestine 28 is illustrated here, this is merely an example, and the present disclosure is established even in a case of an endoscopy of a luminal organ such as an esophagus, a stomach, a duodenum, or a trachea.

The light source device 20, the control device 22, and the medical support device 24 are installed on a wagon 34. A plurality of tables are provided in the wagon 34 along a vertical direction, and the medical support device 24, the control device 22, and the light source device 20 are installed from a lower table to an upper table. In addition, the display device 18 is installed on the uppermost table in the wagon 34.

The control device 22 controls the entire endoscope apparatus 10. The medical support device 24 performs various types of image processing on an image obtained by imaging the intestinal wall 32 with the endoscope 16 under the control of the control device 22.

The display device 18 displays various types of information including the image. Examples of the display device 18 include a liquid crystal display or an EL display. In addition, a tablet terminal with a display may be used instead of the display device 18 or together with the display device 18.

A screen 35 is displayed on the display device 18. The screen 35 includes a plurality of display regions. The plurality of display regions are arranged side by side in the screen 35. In the example shown in FIG. 1, a first display region 36 and a second display region 38 are shown as an example of the plurality of display regions. A size of the first display region 36 is larger than a size of the second display region 38. The first display region 36 is used as a main display region, and the second display region 38 is used as a sub-display region. A size relationship between the first display region 36 and the second display region 38 is not limited to this, and need only be a size relationship that fits on the screen 35. In the first embodiment, the screen 35 is an example of a “first screen” and a “second screen” according to the present disclosure.

An endoscopic moving image 39 is displayed in the first display region 36. The endoscopic moving image 39 is a moving image acquired by imaging the intestinal wall 32 with the endoscope 16 inside the large intestine 28 of the subject 26. In the example shown in FIG. 1, as an example of the endoscopic moving image 39, a moving image in which the intestinal wall 32 is shown is shown.

The intestinal wall 32 shown in the endoscopic moving image 39 includes a lesion 42 (for example, one lesion 42 in the example shown in FIG. 1,) as a region of interest (that is, an observation target region) that is watched by the doctor 12, and the doctor 12 can visually recognize an aspect of the intestinal wall 32 including the lesion 42 through the endoscopic moving image 39. In the first embodiment, the lesion 42 is an example of a “first feature region”, a “second feature region”, a “third feature region”, a “fourth feature region”, and a “lesion” according to the present disclosure.

The lesion 42 has various types, and examples of the type of the lesion 42 include a neoplastic polyp and a non-neoplastic polyp. Examples of the type of the neoplastic polyp include an adenomatous polyp (for example, SSL). Examples of the type of the non-neoplastic polyp include a hamartomatous polyp, a hyperplastic polyp, and an inflammatory polyp. The types illustrated here are types assumed in advance as the types of the lesion 42 in a case where the endoscopy is performed on the large intestine 28, and the types of the lesion 42 may be different depending on the organ on which the endoscopy is performed.

In the first embodiment, for convenience of description, a form example is described in which one lesion 42 is shown in the endoscopic moving image 39, but the present disclosure is not limited to this. The present disclosure is established even in a case where a plurality of the lesions 42 are shown in the endoscopic moving image 39.

In the first embodiment, the lesion 42 is illustrated, but this is merely an example. The region of interest (that is, the observation target region) that is watched by the doctor 12 may be a feature region having some unique feature, such as an organ (for example, a duodenal papilla), a mark, an artificial treatment tool (for example, an artificial clip), a treated region (for example, a region in which a trace of removal of a polyp or the like remains), or the like.

The image displayed in the first display region 36 is one frame 40 included in a moving image configured to include a plurality of frames 40 along a time series. That is, a plurality of frames 40 along the time series are displayed in the first display region 36 at a predetermined frame rate (for example, several tens of frames/second).

Examples of the moving image displayed in the first display region 36 include a moving image of a live view method. The live view method is only an example, and a moving image which is temporarily stored in a memory or the like and then is displayed, such as a moving image of a post view method, may be employed. In addition, each frame included in a recording moving image stored in a memory or the like may be reproduced and displayed on the screen 35 (for example, the first display region 36) as the endoscopic moving image 39.

In the screen 35, the second display region 38 is adjacent to the first display region 36 and is displayed in the lower right in the screen 35 in front view. A display position of the second display region 38 may be anywhere in the screen 35 of the display device 18. However, the display position is preferably displayed at a position comparable to the endoscopic moving image 39.

In the second display region 38, assistance information 44 for assisting the doctor 12 in medical determination or the like in the endoscopy is displayed. The assistance information 44 is information referred to by the doctor 12. Examples of the assistance information 44 include various types of information about the subject 26 in which the endoscope 16 is inserted into the body, and/or various types of information obtained by performing a medical support process described below.

The endoscopy for the large intestine 28 includes an insertion step 41A and a removal step 41B. The insertion step 41A is a step in which the endoscope 16 advances to a far side of the large intestine 28, that is, a step of inserting the endoscope 16 into the large intestine 28 (here, as an example, a step of inserting the endoscope 16 from an anus to ileocecal portion 28A). The removal step 41B is a step in which the endoscope 16 advances to an entrance side of the large intestine 28, that is, a step of removing the endoscope 16 from the large intestine 28 (here, as an example, a step of removing the endoscope 16 from the ileocecal portion 28A to the anus).

In the insertion step 41A, the endoscope 16 is inserted into the large intestine 28 along an insertion path 29A. For example, the insertion path 29A refers to a path from the anus (not shown) to the ileocecal portion 28A. Here, the anus is illustrated as a start point of the insertion path 29A, but this is merely an example. For example, the start point of the insertion path 29A may be a position at which the endoscopy is started in the large intestine 28 (for example, a position at a point in time at which an instruction to start the endoscopy is given).

In the removal step 41B, the endoscope 16 is removed from the large intestine 28 along a removal path 29B. For example, the removal path 29B refers to a path from the ileocecal portion 28A to the anus.

In the insertion step 41A, in a case where the endoscope 16 reaches the ileocecal portion 28A of the large intestine 28 along the insertion path 29A, the process is switched to the removal step 41B from the insertion step 41A, and the endoscope 16 is removed from the large intestine 28 along the removal path 29B. In the insertion step 41A, imaging of the inside of the large intestine 28 is performed by the endoscope apparatus 10. In the removal step 41B, imaging of the inside of the large intestine 28 is performed by the endoscope apparatus 10 in the same manner as in the insertion step 41A. In addition, in the removal step 41B, a medical treatment (for example, discrimination, marking, resection, and/or hemostasis) is performed by the endoscope apparatus 10.

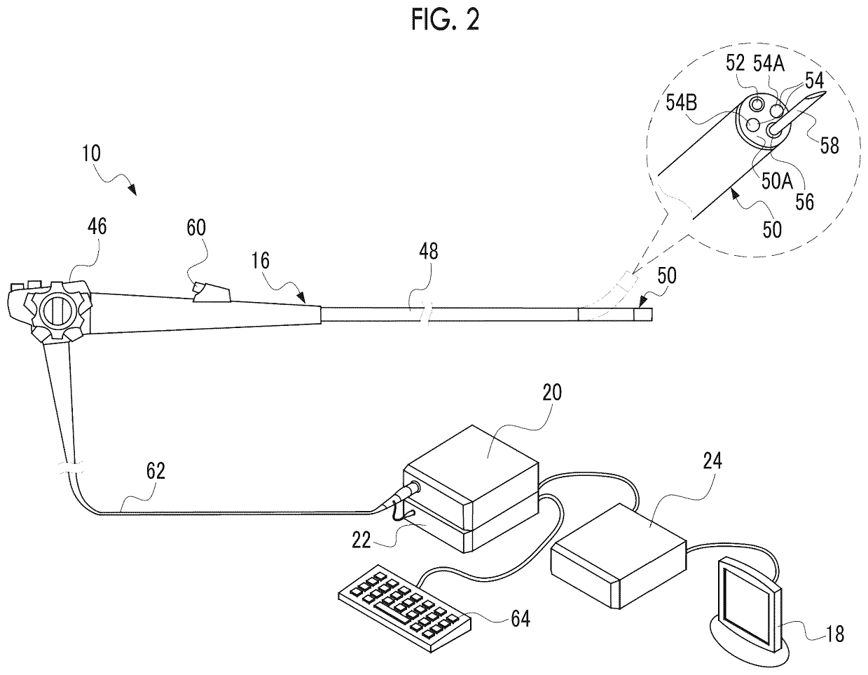

FIG. 2 is a conceptual diagram showing an example of an overall configuration of the endoscope apparatus 10. As shown in FIG. 2, the endoscope 16 comprises an operating part 46 and an insertion part 48. The insertion part 48 is partially bent by operating the operating part 46. The insertion part 48 is inserted into the large intestine 28 while being bent according to a shape of the large intestine 28 (see FIG. 1) in response to the operation of the operating part 46 by the doctor 12 (see FIG. 1).

A camera 52, an illumination device 54, and a treatment tool opening 56 are provided in a distal end part 50 of the insertion part 48. The camera 52 and the illumination device 54 are provided on a distal end surface 50A of the distal end part 50. Here, although a form example is described in which the camera 52 and the illumination device 54 are provided on the distal end surface 50A of the distal end part 50, this is merely an example. The camera 52 and the illumination device 54 may be provided on a side surface of the distal end part 50, so that the endoscope 16 may be configured as a side-viewing endoscope.

The camera 52 is inserted into a body cavity of the subject 26 to image the observation target region. In the first embodiment, the camera 52 acquires the endoscopic moving image 39 by imaging the inside of the body (for example, the inside of the large intestine 28) of the subject 26. Examples of the camera 52 include a CMOS camera. However, this is only an example, and the camera may be other types of camera such as a CCD camera. The camera 52 is an example of a “camera” according to the present disclosure.

The illumination device 54 has illumination windows 54A and 54B. The illumination device 54 emits the light 30 (see FIG. 1) through illumination windows 54A and 54B. Examples of the type of the light 30 emitted from the illumination device 54 include visible light (for example, white light) and invisible light (for example, near-infrared light). In addition, the illumination device 54 emits special light through the illumination windows 54A and 54B. Examples of the special light include light for BLI and/or light for LCI. The camera 52 images the inside of the large intestine 28 by an optical method in a state in which the light 30 is emitted inside the large intestine 28 by the illumination device 54.

The treatment tool opening 56 is an opening through which a treatment tool 58 protrudes from the distal end part 50. In addition, the treatment tool opening 56 is also used as a suction port for sucking blood, internal filth, and the like and a delivery port for sending out a fluid.

A treatment tool insertion port 60 is formed in the operating part 46, and the treatment tool 58 is inserted into the insertion part 48 through the treatment tool insertion port 60. The treatment tool 58 passes through the insertion part 48 and protrudes from the treatment tool opening 56 to the outside. In the example shown in FIG. 2, an aspect is shown in which a puncture needle protrudes from the treatment tool opening 56 as the treatment tool 58. Here, the puncture needle is illustrated as the treatment tool 58, but this is merely an example. The treatment tool 58 may be a grasping forceps, a papillotomy knife, a snare, a catheter, a guide wire, a cannula, and/or a puncture needle with a guide sheath, or the like.

The endoscope 16 is connected to the light source device 20 and the control device 22 via a universal cord 62. The medical support device 24 and a reception device 64 are connected to the control device 22. In addition, the display device 18 is connected to the medical support device 24. That is, the control device 22 is connected to the display device 18 via the medical support device 24.

Here, since the medical support device 24 is illustrated as an externally connected device for expanding a function performed by the control device 22, a form example is described in which the control device 22 and the display device 18 are indirectly connected to each other via the medical support device 24, but this is merely an example. For example, the display device 18 may be directly connected to the control device 22. In this case, for example, the functions of the medical support device 24 may be provided in the control device 22, or the control device 22 may be provided with a function of causing a server (not shown) to execute the same process as the process (for example, a medical support process which will be described below) executed by the medical support device 24, receiving a processing result of the server, and using the processing result.

The reception device 64 receives an instruction from the doctor 12 and outputs the received instruction as an electric signal to the control device 22. Examples of the reception device 64 include a keyboard, a mouse, a touch panel, a foot switch, a microphone, and/or a remote control device.

The control device 22 controls the light source device 20, transmits and receives various signals to and from the camera 52, or transmits and receives various signals to and from the medical support device 24.

The light source device 20 emits light under the control of the control device 22 and supplies the light to the illumination device 54. A light guide is provided in the illumination device 54, and the light supplied from the light source device 20 is emitted from the illumination windows 54A and 54B through the light guide. The control device 22 causes the camera 52 to perform imaging, acquires the endoscopic moving image 39 (see FIG. 1) from the camera 52, and outputs the endoscopic moving image 39 to a predetermined output destination (for example, the medical support device 24).

The medical support device 24 supports medical care (here, as an example, an endoscopy) by performing various types of image processing on the endoscopic moving image 39 input from the control device 22. The medical support device 24 outputs the endoscopic moving image 39 that has been subjected to various types of image processing to a predetermined output destination (for example, the display device 18).

Here, a form example is described in which the endoscopic moving image 39 output from the control device 22 is output to the display device 18 via the medical support device 24, but this is merely an example. For example, the control device 22 and the display device 18 may be connected to each other, and the endoscopic moving image 39 that has been subjected to the image processing by the medical support device 24 may be displayed on the display device 18 via the control device 22.

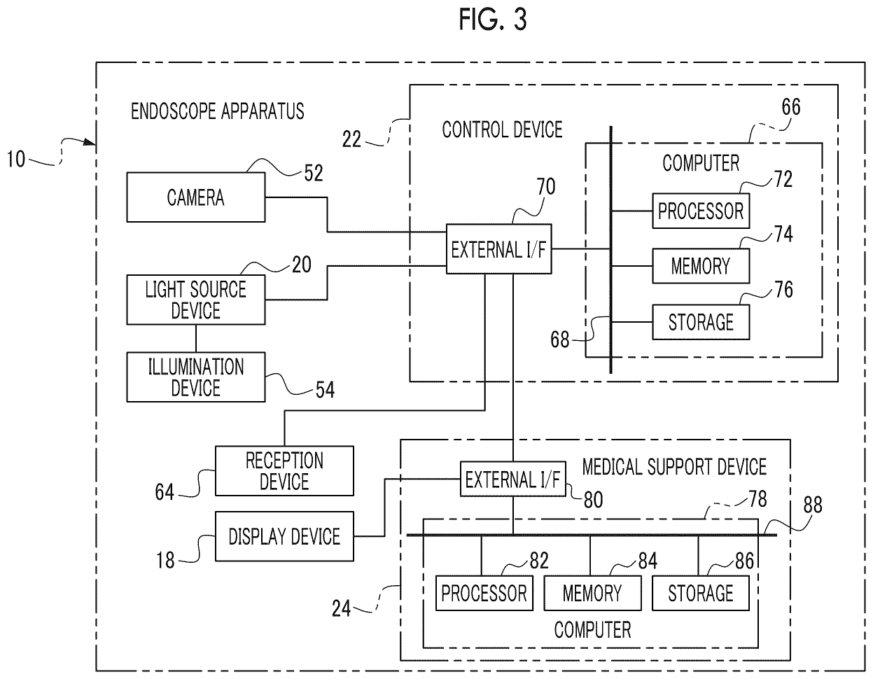

FIG. 3 is a block diagram showing an example of a hardware configuration of an electric system of the endoscope apparatus 10. As shown in FIG. 3, the control device 22 comprises a computer 66, a bus 68, and an external I/F 70. The computer 66 comprises a processor 72, a memory 74, and a storage 76. The processor 72, the memory 74, the storage 76, and the external I/F 70 are connected to the bus 68. The processor 72 controls the entire control device 22. The memory 74 and the storage 76 are used by the processor 72.

The external I/F 70 transmits and receives various types of information between one or more devices (hereinafter, also referred to as “first external devices”) outside the control device 22 and the processor 72.

As one of the first external devices, the camera 52 is connected to the external I/F 70, and the external I/F 70 transmits and receives various types of information between the camera 52 and the processor 72. The processor 72 controls the camera 52 via the external I/F 70. In addition, the processor 72 acquires the endoscopic moving image 39 (see FIG. 1) obtained by imaging the inside of the large intestine 28 (see FIG. 1) by the camera 52 via the external I/F 70.

As one of the first external devices, the light source device 20 is connected to the external I/F 70, and the external I/F 70 transmits and receives various types of information between the light source device 20 and the processor 72. The light source device 20 supplies light to the illumination device 54 under the control of the processor 72. The illumination device 54 performs irradiation with the light supplied from the light source device 20.

As one of the first external devices, the reception device 64 is connected to the external I/F 70. The processor 72 acquires the instruction received by the reception device 64 via the external I/F 70 and performs a process corresponding to the acquired instruction.

The medical support device 24 comprises a computer 78 and an external I/F 80. The computer 78 comprises a processor 82, a memory 84, and a storage 86. The processor 82, the memory 84, the storage 86, and the external I/F 80 are connected to a bus 88. In the first embodiment, the medical support device 24 is an example of a “medical support device” according to the present disclosure, the computer 78 is an example of a “computer” according to the present disclosure, and the processor 82 is an example of a “processor” according to the present disclosure.

Since a hardware configuration (that is, the processor 82, the memory 84, and the storage 86) of the computer 78 is basically the same as the hardware configuration of the computer 66, the hardware configuration of the computer 78 will not be described here.

The external I/F 80 transmits and receives various types of information between one or more devices (hereinafter, also referred to as “second external devices”) outside the medical support device 24 and the processor 82.

As one of the second external devices, the control device 22 is connected to the external I/F 80. In the example shown in FIG. 3, the external I/F 70 of the control device 22 is connected to the external I/F 80. The external I/F 80 transmits and receives various types of information between the processor 82 of the medical support device 24 and the processor 72 of the control device 22. For example, the processor 82 acquires the endoscopic moving image 39 (see FIG. 1) from the processor 72 of the control device 22 via the external I/Fs 70 and 80 and performs various types of image processing on the acquired endoscopic moving image 39.

As one of the second external devices, the display device 18 is connected to the external I/F 80. The processor 82 controls the display device 18 via the external I/F 80 so that various types of information (for example, the endoscopic moving image 39 subjected to various types of image processing) are displayed on the display device 18.

Meanwhile, in the endoscopy, the doctor 12 determines whether or not a medical treatment is necessary for the lesion 42 shown in the endoscopic moving image 39 while confirming the endoscopic moving image 39 via the display device 18, and performs the medical treatment on the lesion 42 in a case where the medical treatment is necessary.

In a general endoscopy, in a case where the doctor 12 finds the lesion 42 via the screen 35 (see FIG. 1) in the insertion step 41A (see FIG. 1), the doctor 12 performs a medical treatment on the lesion 42 found in the insertion step 41A, in the removal step 41B. In this case, the doctor 12 needs to visually specify the lesion 42 found in the insertion step 41A thoroughly in the removal step 41B again.

However, in general, the endoscope 16 is inserted into the large intestine 28 in a state in which air is sucked from the inside of the large intestine 28 in the insertion step 41A, and the endoscope 16 is removed from the large intestine 28 in a state in which air is supplied to the inside of the large intestine 28 in the removal step 41B. Therefore, an appearance of the lesion 42 observed in the insertion step 41A and an appearance of the lesion 42 observed in the removal step 41B may be different from each other even for the same lesion 42. In addition, since a body motion occurs or an orientation of the camera 52 is changed between the insertion step 41A and the removal step 41B, the appearance of the lesion 42 observed in the insertion step 41A and the appearance of the lesion 42 observed in the removal step 41B may be different from each other even for the same lesion 42. In this case, in the removal step 41B, the doctor 12 tends to overlook the lesion 42 found in the insertion step 41A as compared with a case where the appearance of the lesion 42 observed in the insertion step 41A is the same as the appearance of the lesion 42 observed in the removal step 41B.

In order to ensure that the medical treatment is thoroughly performed on each of all the lesions 42 found in the endoscopy, it is very important that the lesion 42 found in the insertion step 41A is visually specified thoroughly in the removal step 41B.

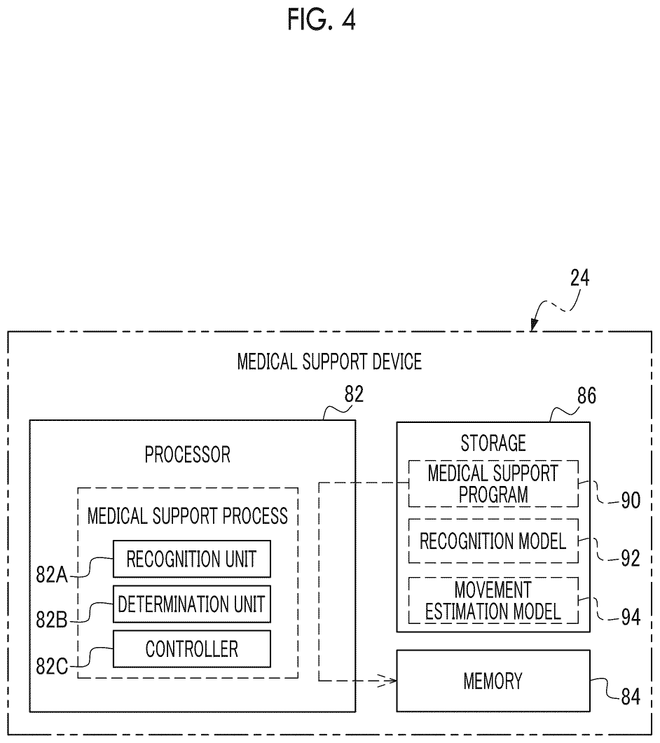

Therefore, in view of such circumstances, in the first embodiment, as shown in FIG. 4 as an example, the medical support process is performed by the processor 82 of the medical support device 24. FIG. 4 is a block diagram showing an example of functions of main units of the processor 82 included in the medical support device 24 and an example of information stored in the storage 86.

A medical support program 90 is stored in the storage 86. The medical support program 90 is an example of a “program” according to the present disclosure. The processor 82 reads out the medical support program 90 from the storage 86 and executes the read-out medical support program 90 on the memory 84 to perform the medical support process. The medical support process is realized by the processor 82 operating as a recognition unit 82A, an estimation unit 82B, and a controller 82C according to the medical support program 90 executed on the memory 84.

The storage 86 stores a recognition model 92 and a movement estimation model 94. Although the details will be described later, the recognition model 92 is used by the recognition unit 82A, and the movement estimation model 94 is used by the estimation unit 82B.

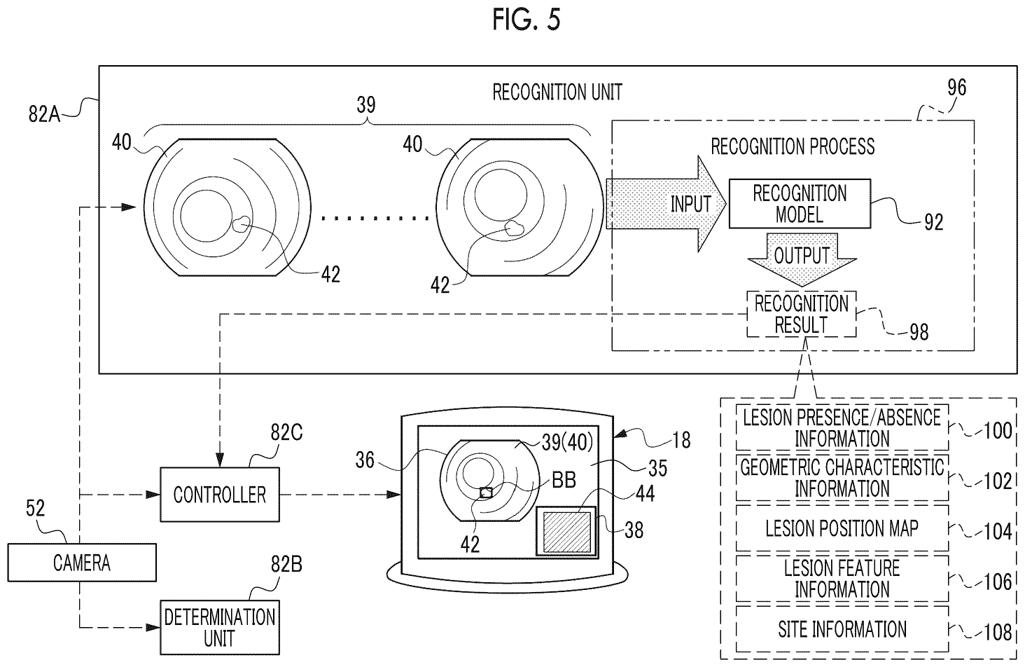

FIG. 5 is a conceptual diagram showing an example of processing contents of the recognition unit 82A, the estimation unit 82B, and the controller 82C. As shown in FIG. 5, the recognition unit 82A, the estimation unit 82B, and the controller 82C acquire each of the plurality of frames 40 along the time series included in the endoscopic moving image 39, which is generated by being captured by the camera 52 at an imaging frame rate (for example, several tens of frames/second), from the camera 52 in units of one frame along the time series. Timings at which the recognition unit 82A, the estimation unit 82B, and the controller 82C acquire the frame 40 are synchronized.

The controller 82C outputs the endoscopic moving image 39 to the display device 18. For example, the controller 82C displays the endoscopic moving image 39 in the first display region 36 as a live view image. That is, each time the frame 40 is acquired from the camera 52, the controller 82C displays the acquired frame 40 in the first display region 36 in order at a display frame rate (for example, several tens of frames/second). In addition, the controller 82C displays the assistance information 44 in the second display region 38. In addition, for example, the controller 82C updates the display content (for example, the assistance information 44) of the second display region 38 according to the display content of the first display region 36.

The recognition unit 82A recognizes the lesion 42 in the endoscopic moving image 39 based on the endoscopic moving image 39 acquired from the camera 52. That is, the recognition unit 82A recognizes the lesion 42 shown in the frame 40 by sequentially performing a recognition process 96 on each of the plurality of frames 40 along the time series included in the endoscopic moving image 39 acquired from the camera 52. For example, the recognition unit 82A recognizes geometric characteristics (for example, a position and a shape) of the lesion 42, a kind of the lesion 42, a type of the lesion 42 (for example, a pedunculated type, a subpedunculated type, a sessile type, a surface raised type, a surface flat type, a surface depressed type, and the like), and the like.

Further, the recognition unit 82A recognizes a site in the endoscopic moving image 39 (hereinafter, simply referred to as a “site”) based on the endoscopic moving image 39 acquired from the camera 52. Here, the site refers to a site of the large intestine 28. Examples of the site of the large intestine 28 include an anal canal, a lower rectum, an upper rectum, a sigmoid colon, a descending colon, a transverse colon, an ascending colon, and an ileocecal portion.

The recognition process 96 is performed on the acquired frame 40 each time the frame 40 is acquired by the recognition unit 82A. The recognition process 96 is a process of recognizing the lesion 42 and the site by a method using AI. Here, as the recognition process 96, a process using the recognition model 92 is performed. The recognition model 92 is a trained model for object recognition in a bounding box method using AI.

The recognition model 92 is optimized by performing machine learning on a neural network using first training data. The first training data is a data set including a plurality of data (that is, a plurality of frames of data) in which first example data and first correct answer data are associated with each other.

The first example data is an image assuming the frame 40. First examples of the image assuming the frame 40 include an image obtained by actually imaging the inside of the large intestine with the camera. Second examples of the image assuming the frame 40 include an image virtually created. The first correct answer data is correct answer data (that is, an annotation) for the first example data. Here, as an example of the first correct answer data, an annotation for specifying geometric characteristics of a lesion shown in an image used as the first example data, a kind of the lesion, a type of the lesion, and a site is used.

The recognition unit 82A acquires the frame 40 from the camera 52 and inputs the acquired frame 40 to the recognition model 92. As a result, the recognition model 92 recognizes the lesion 42 and the site shown in the input frame 40 each time the frame 40 is input, and outputs a recognition result 98.

The recognition unit 82A outputs the recognition result 98 itself to the display device 18 or the like in synchronization with the display of the frame 40 in the first display region 36, or outputs information generated based on the recognition result 98 to the display device 18 or the like in synchronization with the display of the frame 40 in the first display region 36. On the screen 35 (for example, the first display region 36 and/or the second display region 38) of the display device 18, the recognition result 98 itself is displayed, or the information generated based on the recognition result 98 is displayed. The display of the recognition result 98 itself and the display of the information generated based on the recognition result 98 are updated in synchronization with a display timing of the endoscopic moving image 39 displayed in the first display region 36. That is, the display of the recognition result 98 itself and the display of the information generated based on the recognition result 98 are updated according to a display frame rate applied to the first display region 36.

The recognition result 98 includes lesion presence/absence information 100. The lesion presence/absence information 100 is information indicating whether or not the lesion 42 is shown in the frame 40 input to the recognition model 92. In addition, the recognition result 98 includes geometrical characteristic information 102, a lesion position map 104, lesion feature information 106, and the like.

The geometric characteristic information 102 is information (for example, coordinates) for specifying a shape and a position of the lesion 42 in the frame 40. The lesion position map 104 is a map for specifying the position of the lesion 42 in the frame 40. The lesion feature information 106 is information for specifying the kind of the lesion 42 shown in the frame 40 input to the recognition model 92 and the type of the lesion 42.

Geometric characteristics (for example, a shape and a size of an outer contour) of the lesion position map 104 correspond to geometric characteristics (for example, a shape and a size of an outer contour) of the frame 40. The lesion position map 104 includes a bounding box BB. The bounding box BB is a rectangular frame (for example, a rectangular border circumscribing an image region showing the lesion 42) for specifying a position recognized by the recognition model 92 as the position of the lesion 42 in the frame 40, which is shown in the frame 40. In the example shown in FIG. 5, the bounding box BB is displayed to be superimposed at a position where the lesion 42 in the frame 40 displayed in the first display region 36 is shown. The display of the bounding box BB is updated in synchronization with a display timing of the endoscopic moving image 39 displayed in the first display region 36.

The lesion position map 104 may be displayed on the screen 35 (for example, the second display region 38) by the controller 82C as one of pieces of assistance information 44. In this case, the lesion position map 104 displayed on the screen 35 is updated according to a display frame rate applied to the first display region 36. With such a configuration, the doctor 12 can ascertain an approximate position of the lesion 42 in the endoscopic moving image 39 displayed in the first display region 36 by referring to the lesion position map 104 displayed in the second display region 38 while observing the endoscopic moving image 39 displayed in the first display region 36.

The controller 82C may display the lesion feature information 106 on the screen 35. For example, the lesion feature information 106 may be displayed in the second display region 38 as one of pieces of the assistance information 44, or may be displayed in a location other than the second display region 38 in the screen 35. In addition, the controller 82C may display an identifier (for example, a mark) instead of the bounding box BB on the screen 35 (in the example shown in FIG. 5, the first display region 36 in the screen 35). In this case, for example, the controller 82C need only specify the shape and the position of the lesion 42 in the frame 40 from the geometrical characteristic information 102 and display the identifier instead of the bounding box BB at the specified shape and position.

The recognition result 98 also includes site information 108. The site information 108 is information for specifying a site shown in the frame 40.

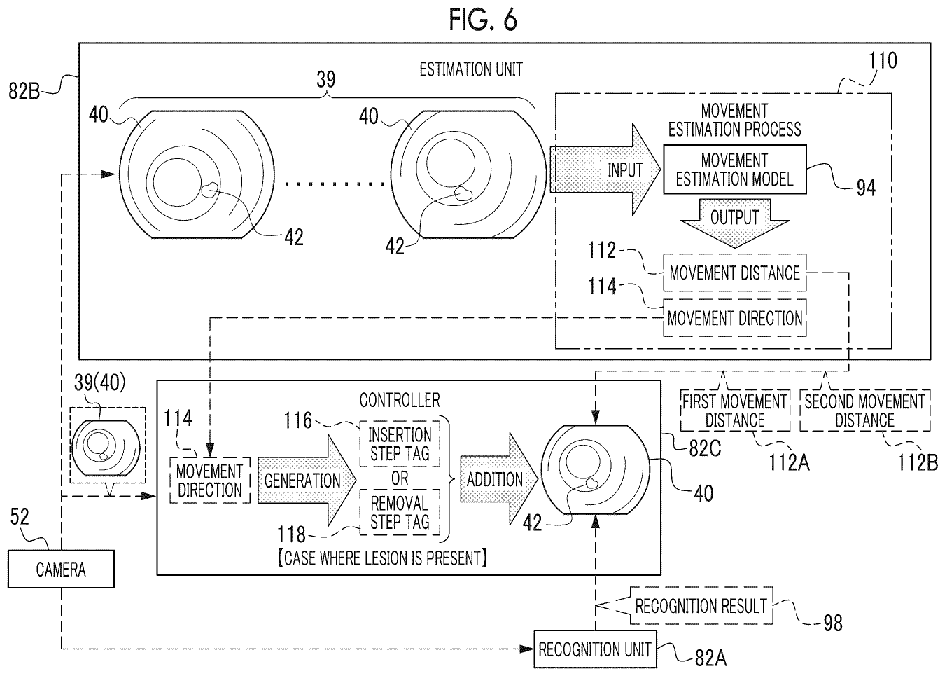

FIG. 6 is a conceptual diagram showing an example of processing contents of the estimation unit 82B and the controller 82C in the insertion step 41A or the removal step 41B. As shown in FIG. 6, the estimation unit 82B recognizes a movement aspect of the camera 52 inserted into the large intestine 28 by performing a movement estimation process 110 based on the endoscopic moving image 39 acquired from the camera 52. Here, the movement aspect refers to, for example, a movement distance 112 and a movement direction 114.

In the first embodiment, information for specifying the position of the camera 52 inserted into the large intestine 28 within the large intestine 28 is defined by the movement distance 112. The movement distance 112 is an example of “position specifying information” and a “movement distance” according to the present disclosure. The information for specifying the position of the camera 52 inserted into the large intestine 28 within the large intestine 28 may be defined by the movement distance 112, the movement direction 114, and the like.

The movement distance 112 is a movement distance of the camera 52 inside the large intestine 28. The movement distance of the camera 52 inside the large intestine 28 refers to, for example, a movement distance of the camera 52 at a point in time at which the camera 52 performs imaging in a period from a start of insertion of the camera 52 into the large intestine 28 to a completion of removal of the camera 52 from the large intestine 28 (for example, a distance moved by the camera 52 from a position (for example, a position of the anus) at which the camera 52 is inserted into the large intestine 28 to a current position). In the first embodiment, for convenience of description, the description will be made on the premise that the camera 52 advances to the ileocecal portion 28A (see FIG. 1) along the insertion path 29A (see FIG. 1) without returning in the insertion step 41A (see FIG. 1), and the camera 52 advances to the anal side along the removal path 29B (see FIG. 1) without returning to the ileocecal portion 28A side in the removal step 41B (see FIG. 1).

The movement direction 114 is an insertion direction or a removal direction. The insertion direction refers to a direction in which the camera 52 is inserted inside the large intestine 28 (that is, a direction in which the camera 52 advances to the ileocecal portion 28A along the insertion path 29A). The removal direction refers to a direction in which the camera 52 is removed from the large intestine 28 (that is, a direction in which the camera 52 advances to the anal side along the removal path 29B).

The movement estimation process 110 is performed by the estimation unit 82B each time the frame 40 is acquired. The movement estimation process 110 is a process of estimating the movement distance 112 and the movement direction 114 by a method using AI. Here, as the movement estimation process 110, a process using the movement estimation model 94 is performed. The movement estimation model 94 is a trained model that estimates the movement distance 112 and the movement direction 114 based on the input plurality of frames 40 along the time series.

The movement estimation model 94 is optimized by performing machine learning on a neural network using second training data. The second training data is a data set including a plurality of data in which second example data and second correct answer data are associated with each other.

The second example data is an image assuming a plurality of frames 40 along a time series (for example, two frames 40 along a time series). First examples of the image assuming a plurality of frames 40 along a time series include a plurality of images along a time series obtained by actually imaging the inside of the large intestine with the camera. Second examples of the image assuming the frames 40 include a plurality of images virtually created by assuming a plurality of images along a time series obtained by actually imaging the inside of the large intestine with the camera. The second correct answer data is correct answer data (that is, an annotation) for the second example data. Here, as the second correct answer data, for example, an annotation for specifying the movement distance of the camera inserted into the large intestine inside the large intestine and the movement direction of the camera inside the large intestine is used.

The estimation unit 82B acquires a plurality of frames 40 along the time series from the camera 52, and inputs the acquired plurality of frames 40 to the movement estimation model 94. Accordingly, each time the plurality of frames 40 along the time series are input, the movement estimation model 94 estimates the movement distance 112 and the movement direction 114 based on the input plurality of frames 40 and outputs the estimated movement distance 112 and movement direction 114 to the controller 82C.

Here, although a form example has been described in which the movement distance 112 and the movement direction 114 are estimated by the movement estimation model 94, the present disclosure is not limited to this. For example, the movement distance 112 and/or the movement direction 114 may be estimated based on an optical flow calculated from the plurality of frames 40 along the time series. In addition, the movement distance 112 and/or the movement direction 114 may be estimated based on both the movement estimation model 94 and the optical flow.

The controller 82C acquires the recognition result 98 from the recognition unit 82A, and determines whether or not the lesion 42 is shown in the frame 40 input to the recognition model 92 by referring to the lesion presence/absence information 100 included in the acquired recognition result 98. Here, in a case where the lesion 42 is shown in the frame 40 that is input to the recognition model 92 in order to obtain the recognition result 98 acquired from the recognition unit 82A by the controller 82C, the controller 82C generates an insertion step tag 116 or a removal step tag 118 based on the movement direction 114 input from the estimation unit 82B. The insertion step tag 116 refers to a tag that is an identifier for identifying the frame 40 obtained by imaging a region including the lesion 42 (that is, a region of a part of the entire region of the intestinal wall 32 that falls within an angle of view of the camera 52 and includes the lesion 42) by the camera 52 in the insertion step 41A. The removal step tag 118 refers to a tag that is an identifier for identifying the frame 40 obtained by imaging a region including the lesion 42 (that is, a region of a part of the entire region of the intestinal wall 32 that falls within an angle of view of the camera 52 and includes the lesion 42) by the camera 52 in the removal step 41B.

In a case where the controller 82C generates the insertion step tag 116 or the removal step tag 118 in this way, first, the controller 82C determines whether the movement direction 114 input from the estimation unit 82B is an insertion direction or a removal direction. Moreover, in a case where the movement direction 114 input from the estimation unit 82B is the insertion direction, the controller 82C generates the insertion step tag 116, and in a case where the movement direction 114 input from the estimation unit 82B is the removal direction, the controller 82C generates the removal step tag 118. In the first embodiment, the insertion step tag 116 is an example of an “insertion step identifier” according to the present disclosure, and the removal step tag 118 is an example of a “removal step identifier” according to the present disclosure.

The controller 82C adds the insertion step tag 116 or the removal step tag 118, the movement distance 112, and the recognition result 98 to the frame 40 which is acquired from the camera 52 and in which the lesion 42 is shown. The frame 40 to which the insertion step tag 116 or the removal step tag 118, the movement distance 112, and the recognition result 98 are added is the same frame 40 as the latest frame 40 input to the movement estimation model 94 for estimating the movement direction 114 used to generate the insertion step tag 116 or the removal step tag 118. The movement distance 112 added to the frame 40 is the movement distance 112 estimated by the movement estimation model 94 together with the movement direction 114 used to generate the insertion step tag 116 or the removal step tag 118. The recognition result 98 added to the frame 40 is the recognition result 98 used by the controller 82C to determine whether or not the lesion 42 is present.

The movement distance 112 estimated by the movement estimation model 94 together with the movement direction 114 used to generate the insertion step tag 116 is added to the frame 40 as a first movement distance 112A. The movement distance 112 estimated by the movement estimation model 94 together with the movement direction 114 used to generate the removal step tag 118 is added to the frame 40 as a second movement distance 112B.

Here, the first movement distance 112A is an example of “insertion position specifying information” according to the present disclosure, and the second movement distance 112B is an example of “removal position specifying information” according to the present disclosure.

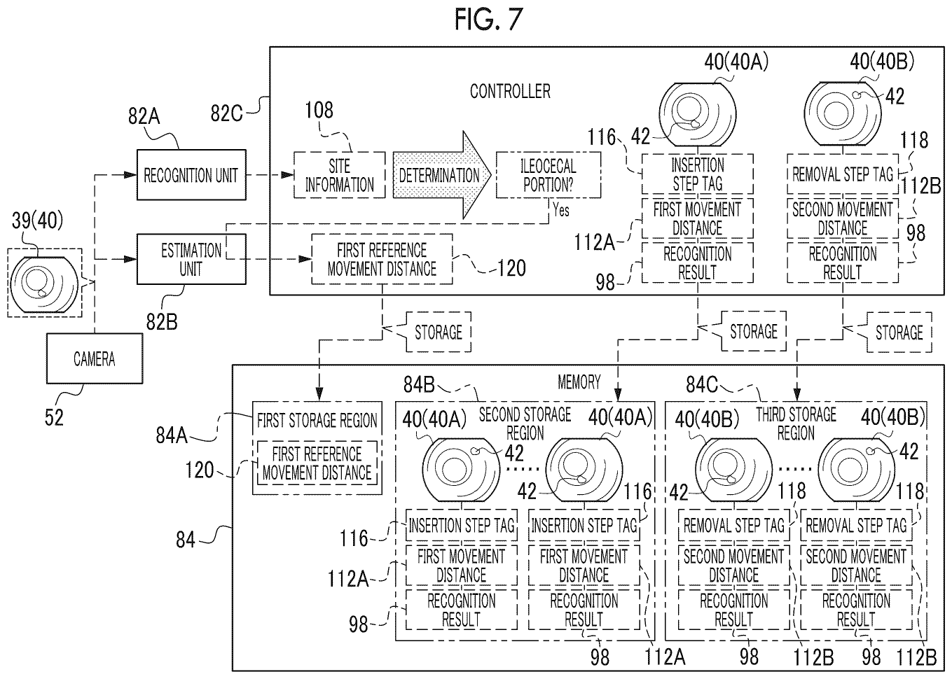

FIG. 7 is a conceptual diagram showing an example of an aspect in which the controller 82C stores various kinds of information in the memory 84. As shown in FIG. 7, the memory 84 includes a first storage region 84A, a second storage region 84B, and a third storage region 84C. The controller 82C stores an insertion step frame 40A in the second storage region 84B in time series. The insertion step frame 40A refers to the frame 40 obtained by imaging a region including the lesion 42 in the large intestine 28 by the camera 52 in the insertion step 41A (in the example shown in FIG. 7, the frame 40 to which the insertion step tag 116, the first movement distance 112A, and the recognition result 98 are added). In addition, the controller 82C stores a removal step frame 40B in the third storage region 84C in time series. The removal step frame 40B refers to the frame 40 obtained by imaging the region including the lesion 42 in the large intestine 28 by the camera 52 in the removal step 41B (in the example shown in FIG. 7, the frame 40 to which the removal step tag 118, the second movement distance 112B, and the recognition result 98 are added). Here, the insertion step frame 40A is an example of an “insertion step frame” according to the present disclosure, and the removal step frame 40B is an example of a “removal step frame” according to the present disclosure.

The controller 82C acquires the site information 108 from the recognition unit 82A. Then, the controller 82C determines whether or not the camera 52 has reached the ileocecal portion 28A (see FIG. 1) by referring to the site information 108. Here, a form example has been described in which the determination is made as to whether or not the camera 52 has reached the ileocecal portion 28A by referring to the site information 108, but this is merely an example. For example, it may be determined whether or not the camera 52 has reached the ileocecal portion 28A by referring to the site information 108 and a plurality of the movement distances 112 along the time series. That is, in a case where the ileocecal portion 28A is specified by the site information 108, and a plurality of the movement distances 112 (for example, for several tens of frames) along the time series are not changed, it may be determined that the camera 52 has reached the ileocecal portion 28A. In addition, in a case where the ileocecal portion 28A is specified by the site information 108 and a plurality of the movement distances 112 along the time series fall within an allowable error range (for example, a range of ±several millimeters) determined in advance as a range of the movement distance 112 in which it can be determined that the camera 52 is stationary, it may be determined that the camera 52 has reached the ileocecal portion 28A.

In a case where the camera 52 has reached the ileocecal portion 28A, the controller 82C acquires the movement distance 112 estimated by the estimation unit 82B (for example, the movement distance 112 on the insertion path 29A (see FIG. 1) from the anus to the ileocecal portion 28A) as a first reference movement distance 120 from the estimation unit 82B. The controller 82C stores the first reference movement distance 120 in the first storage region 84A.

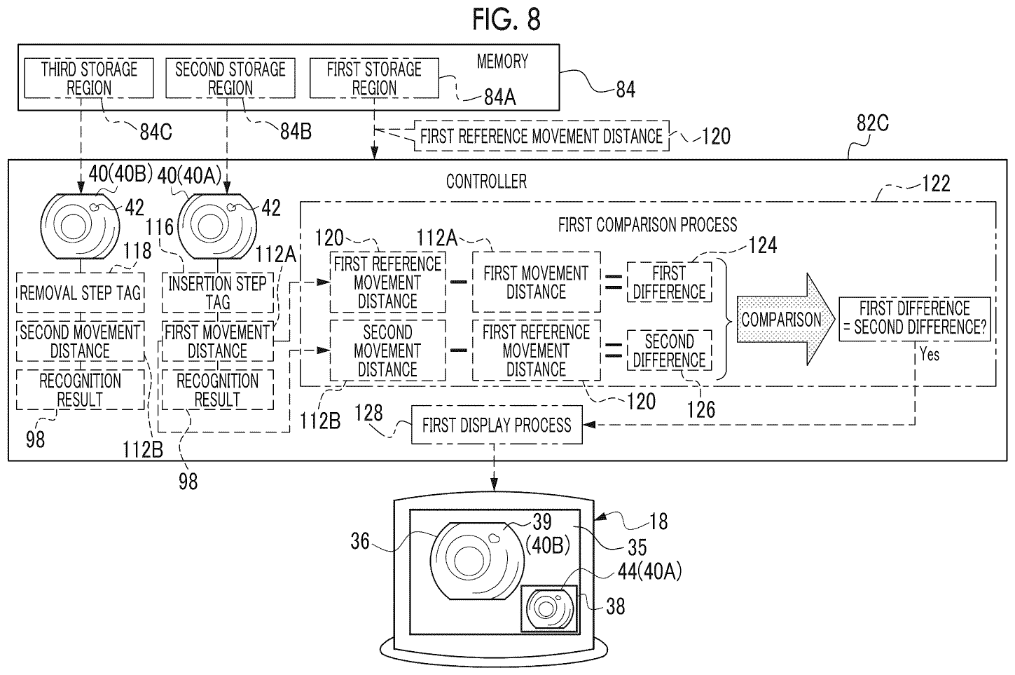

FIG. 8 is a conceptual diagram showing an example of processing contents performed by the controller 82C in the removal step 41B based on the first reference movement distance 120 stored in the first storage region 84A, the first movement distance 112A added to the insertion step frame 40A stored in the second storage region 84B, and the second movement distance 112B added to the removal step frame 40B stored in the third storage region 84C.

As shown in FIG. 8, the controller 82C acquires the first reference movement distance 120 from the first storage region 84A. In addition, the controller 82C acquires the first movement distance 112A added to the insertion step frame 40A from the second storage region 84B. Further, the controller 82C acquires the second movement distance 112B added to the latest removal step frame 40B from the third storage region 84C. Then, the controller 82C performs a first comparison process 122 based on the first reference movement distance 120, the first movement distance 112A, and the second movement distance 112B.

In the first comparison processing 122, a first difference 124 is calculated. The first difference 124 refers to a difference between the first reference movement distance 120 and the first movement distance 112A. The first difference 124 is calculated for each first movement distance 112A added to each of all the insertion step frames 40A stored in the second storage region 84B. In addition, here, as an example of the difference between the first reference movement distance 120 and the first movement distance 112A, a value obtained by subtracting the first movement distance 112A from the first reference movement distance 120 is used.

In the first comparison process 122, a second difference 126 is calculated. The second difference 126 refers to a difference between the second movement distance 112B added to the latest removal step frame 40B stored in the third storage region 84C and the first reference movement distance 120. Here, as an example of the difference between the second movement distance 112B and the first reference movement distance 120, a value obtained by subtracting the first reference movement distance 120 from the second movement distance 112B is used.

In the example shown in FIG. 8, a form example has been described in which the value obtained by subtracting the first movement distance 112A from the first reference movement distance 120 is used as the first difference 124, and the value obtained by subtracting the second movement distance 112B from the first reference movement distance 120 is used as the second difference 126. However, this is merely an example. For example, the first difference 124 may be an absolute value of the difference between the first reference movement distance 120 and the first movement distance 112A, and the second difference 126 may be an absolute value of the difference between the first reference movement distance 120 and the second movement distance 112B. In addition, the first difference 124 may be a value obtained by subtracting the first reference movement distance 120 from the first movement distance 112A, and the second difference 126 may be a value obtained by subtracting the second movement distance 112B from the first reference movement distance 120.

In the first comparison process 122, it is determined whether or not each first difference 124 calculated for each first movement distance 112A added to each of all the insertion step frames 40A stored in the second storage region 84B matches the second difference 126. Here, the term “match” means match including an error that is generally allowed in the technical field to which the present disclosure belongs and does not deviate from the gist of the present disclosure, in addition to perfect match.

In a case where it is determined that the first difference 124 and the second difference 126 match, the controller 82C executes a first specifying process for specifying an identity between the lesion 42 shown in the insertion step frame 40A to which the first movement distance 112A used to calculate the first difference 124 is added and the lesion 42 shown in the removal step frame 40B to which the second movement distance 112B used to calculate the second difference 126 that matches the first difference 124 is added. In the example shown in FIG. 8, a first display process 128 is shown as an example of a process included in the first specifying process. The first display process 128 is an example of a “first specifying process” and a “first display process” according to the present disclosure.

The first display process 128 is a process of displaying the insertion step frame 40A and the removal step frame 40B on the screen 35 in a comparable state in a case where a level of the identity between the lesion 42 shown in the insertion step frame 40A and the lesion 42 shown in the removal step frame 40B is equal to or higher than a predetermined level. Here, the predetermined level is an example of a “first predetermined level” and a “second predetermined level” according to the present disclosure. In the first embodiment, a case where the level of the identity between the lesion 42 shown in the insertion step frame 40A and the lesion 42 shown in the removal step frame 40B is equal to or higher than the predetermined level indicates a case where the first difference 124 and the second difference 126 match each other.

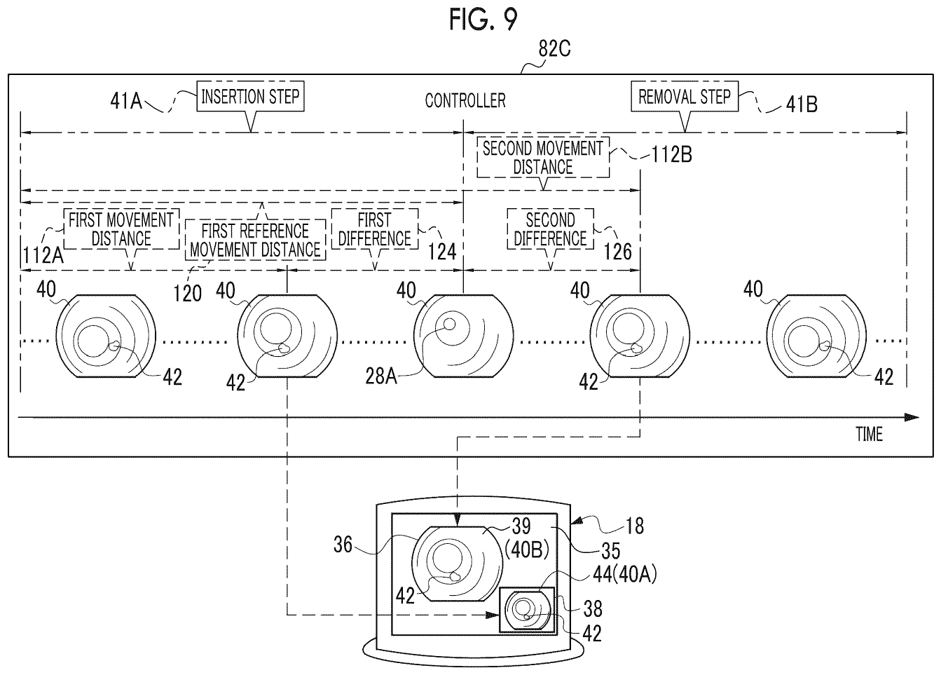

For example, as shown in FIG. 9, in a case where the first difference 124 corresponding to a distance from the lesion 42 found in the insertion step 41A to the ileocecal portion 28A and the second difference 126 corresponding to a distance from the ileocecal portion 28A to the lesion 42 found in the removal step 41B match, the controller 82C determines that the level of the identity between the lesion 42 shown in the insertion step frame 40A and the lesion 42 shown in the removal step frame 40B is equal to or higher than the predetermined level.

As shown in FIGS. 8 and 9, in the first display process 128, the removal step frame 40B to which the second movement distance 112B used to calculate the second difference 126 matching the first difference 124 is added is displayed in the first display region 36, and the insertion step frame 40A to which the first movement distance 112A used to calculate the first difference 124 is added is displayed in the second display region 38. As a result, the removal step frame 40B displayed in the first display region 36 and the insertion step frame 40A displayed in the second display region 38 are in a visually comparable state. Further, the lesion 42 can be visually specified from the removal step frame 40B displayed in the first display region 36, and the lesion 42 can be visually specified from the insertion step frame 40A displayed in the second display region 38. Therefore, the lesion 42 shown in the removal step frame 40B displayed in the first display region 36 and the lesion 42 shown in the insertion step frame 40A displayed in the second display region 38 are in a visually comparable state. Here, the removal step frame 40B displayed in the first display region 36 is an example of a “second image” according to the present disclosure, and the insertion step frame 40A displayed in the second display region 36 is an example of a “first image” according to the present disclosure.

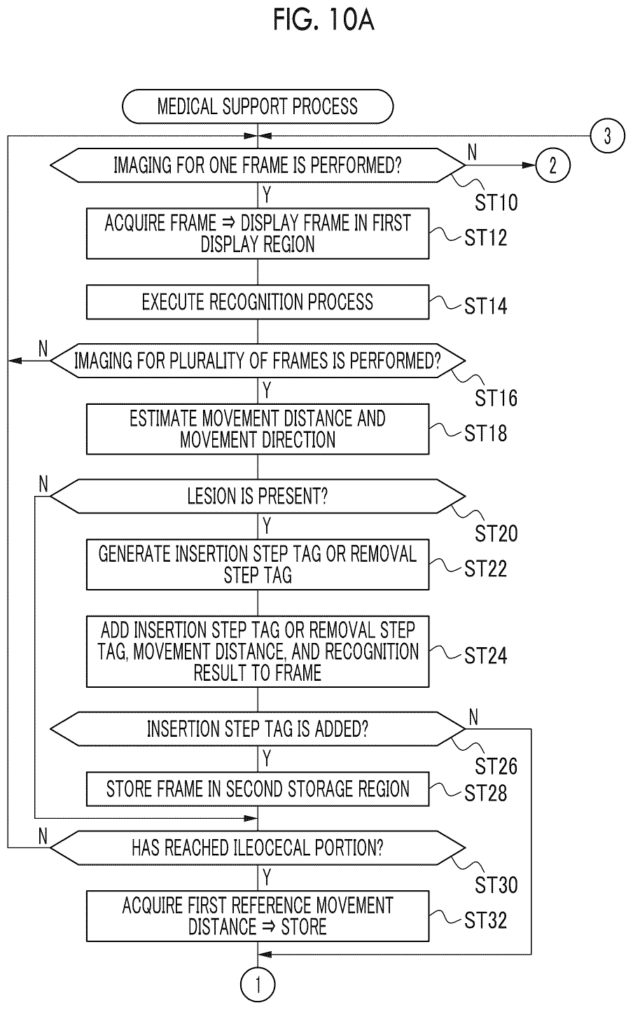

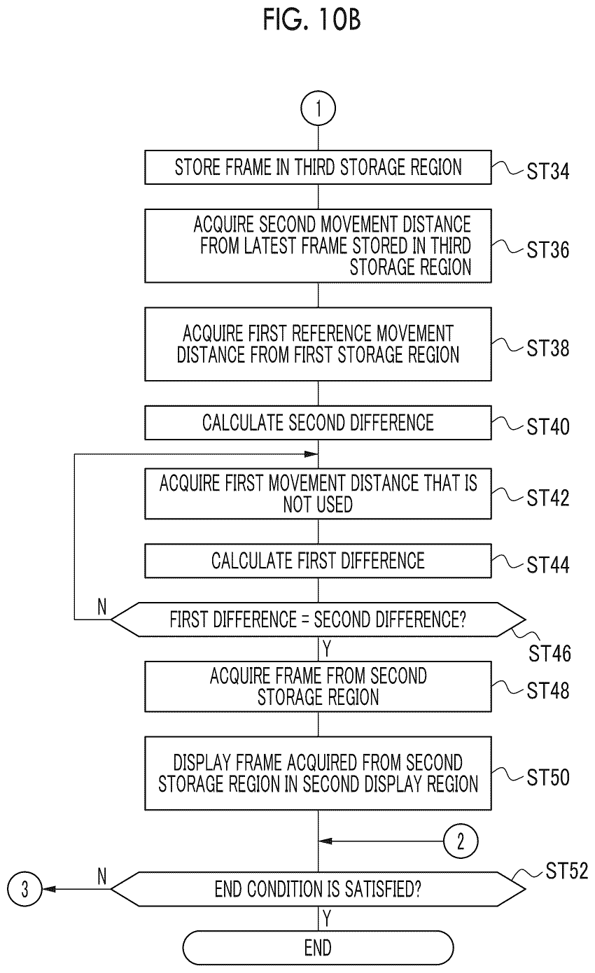

Next, an operation of a part of the endoscope apparatus 10 according to the present disclosure will be described with reference to FIGS. 10A and 10B. A flow of a medical support process shown in FIGS. 10A and 10B is an example of a “medical support method” according to the present disclosure.

In addition, in the following, the description will be made on the premise that the inside of the large intestine 28 is irradiated with the same type of light (for example, light for BLI and/or light for LCI) in the insertion step 41A and the removal step 41B, and a region (for example, the intestinal wall 32) irradiated with the light is imaged by the camera 52.

In the medical support process shown in FIG. 10A, first, in step ST10, the recognition unit 82A determines whether or not imaging for one frame is performed by the camera 52 in the large intestine 28. In step ST10, in a case where the imaging for one frame is not performed by the camera 52 in the large intestine 28, a negative determination is made, and the medical support process proceeds to step ST52 shown in FIG. 10B. In step ST10, in a case where the imaging for one frame is performed by the camera 52 in the large intestine 28, a positive determination is made, and the medical support process proceeds to step ST12.

In step ST12, the recognition unit 82A, the estimation unit 82B, and the controller 82C acquire the frame 40 obtained by imaging the large intestine 28 by the camera 52. Then, the controller 82C displays the frame 40 in the first display region 36. In a case where the frame 40 is already displayed in the first display region 36, the controller 82C updates the frame 40 displayed in the first display region 36. That is, by repeatedly executing the process in step ST12, the frame 40 is displayed in the first display region 36 by the live view method. After the process in step ST12 is executed, the medical support process proceeds to step ST14.

In step ST14, the recognition unit 82A executes the recognition process 96 on the frame 40 acquired in step ST12. After the process in step ST14 is executed, the medical support process proceeds to step ST16.

In step ST16, the estimation unit 82B determines whether or not imaging for a plurality of frames is performed by the camera 52. In step ST16, in a case where the imaging for a plurality of frames is not performed by the camera 52, a negative determination is made, and the medical support process proceeds to step ST10. In step ST16, in a case where the imaging for a plurality of frames is performed by the camera 52, a positive determination is made, and the medical support process proceeds to step ST18.

In step ST18, the estimation unit 82B inputs the plurality of frames 40 along the time series to the movement estimation model 94 to cause the movement estimation model 94 to estimate the movement distance 112 and the movement direction 114. After the process in step ST18 is executed, the medical support process proceeds to step ST20.

In step ST20, the recognition unit 82A determines whether or not the lesion 42 is included in the frame 40 acquired in step ST12 by referring to the lesion presence/absence information 100 included in the recognition result 98 obtained by executing the recognition process 96 in step ST14. In step ST20, in a case where the lesion 42 is not included in the frame 40 acquired in step ST12, a negative determination is made, and the medical support process proceeds to step ST30. In step ST20, in a case where the lesion 42 is included in the frame 40 acquired in step ST12, a positive determination is made, and the medical support process proceeds to step ST22.

In step ST22, the controller 82C generates the insertion step tag 116 or the removal step tag 118 based on the movement direction 114 estimated by the estimation unit 82B in step ST18. That is, in a case where the movement direction 114 is the insertion direction, the insertion step tag 116 is generated, and in a case where the movement direction 114 is the removal direction, the removal step tag 118 is generated. After the process in step ST22 is executed, the medical support process proceeds to step ST24.

In step ST24, the controller 82C adds the insertion step tag 116 or the removal step tag 118 generated in step ST22, the movement distance 112 estimated in step ST18, and the recognition result 98 obtained by executing the recognition process 96 in step ST14 to the frame 40 acquired in step ST12. For example, in a case where the movement direction 114 estimated in step ST18 is the insertion direction, the controller 82C adds the insertion step tag 116, adds the movement distance 112 estimated in step ST18 as the first movement distance 112A, and adds the recognition result 98, to the frame 40 acquired in step ST12, that is, the insertion step frame 40A. In addition, for example, in a case where the movement direction 114 estimated in step ST18 is the removal direction, the controller 82C adds the removal step tag 118, adds the movement distance 112 estimated in step ST18 as the second movement distance 112B, and adds the recognition result 98, to the frame 40 acquired in step ST12, that is, the removal step frame 40B. After the process in step ST24 is performed, the medical support process proceeds to step ST26.

In step ST26, the controller 82C determines whether or not the insertion step tag 116 is added to the frame 40. In step ST26, in a case where the insertion step tag 116 is not added to the frame 40 (that is, in a case where the removal step tag 118 is added to the frame 40), a negative determination is made, and the medical support process proceeds to step ST34 shown in FIG. 10B. In step ST26, in a case where the insertion step tag 116 is added to the frame 40, a positive determination is made, and the medical support process proceeds to step ST28.

In step ST28, the controller 82C stores the insertion step frame 40A to which the insertion step tag 116, the movement distance 112 as the first movement distance 112A, and the recognition result 98 are added, in the second storage region 84B in time series. After the process in step ST28 is performed, the medical support process proceeds to step ST30.

In step ST30, the controller 82C determines whether or not the camera 52 has reached the ileocecal portion 28A by referring to the site information 108 included in the recognition result 98 obtained by executing the recognition process 96 in step ST14. In step ST30, in a case where the camera 52 has not reached the ileocecal portion 28A, a negative determination is made, and the medical support process proceeds to step ST10. In step ST30, in a case where the camera 52 has reached the ileocecal portion 28A, a positive determination is made, and the medical support process proceeds to step ST32.

In step ST32, the controller 82C stores the movement distance 112 estimated in step ST18 in the first storage region 84A as the first reference movement distance 120. After the process in step ST32 is executed, the medical support process proceeds to step ST34 shown in FIG. 10B.

In step ST34 shown in FIG. 10B, the controller 82C stores the removal step frame 40B to which the removal step tag 118, the movement distance 112 as the second movement distance 112B, and the recognition result 98 are added, in the third storage region 84C in time series. After the process in step ST34 is executed, the medical support process proceeds to step ST36.

In step ST36, the controller 82C acquires the second movement distance 112B from the latest removal step frame 40B stored in the third storage region 84C. After the process in step ST34 is executed, the medical support process proceeds to step ST38.

In step ST38, the controller 82C acquires the first reference movement distance 120 from the first storage region 84A. After the process in step ST38 is performed, the medical support process proceeds to step ST40.

In step ST40, the controller 82C calculates the difference between the second movement distance 112B acquired in step 38 and the first reference movement distance 120 acquired in step ST38, that is, the second difference 126. After the process in step ST40 is performed, the medical support process proceeds to step ST42.

In step ST42, the controller 82C acquires the first movement distance 112A that is not used in step ST42. After the process in step ST42 is executed, the medical support process proceeds to step ST44.

In step ST44, the controller 82C calculates the difference between the first movement distance 112A acquired in step ST42 and the first reference movement distance 120 acquired in step ST38, that is, the first difference 124. After the process in step ST44 is executed, the medical support process proceeds to step ST46.

In step ST46, the controller 82C determines whether or not the first difference 124 calculated in step ST44 and the second difference 126 calculated in step ST40 match. In step ST46, in a case where the first difference 124 and the second difference 126 do not match, a negative determination is made, and the medical support process proceeds to step ST42. In step ST46, in a case where the first difference 124 and the second difference 126 match, a positive determination is made, and the medical support process proceeds to step ST48.

In step ST48, the controller 82C acquires the insertion step frame 40A to which the first movement distance 112A used to calculate the first difference 124 matching the second difference 126 is added, from the second storage region 84B. After the process in step ST48 is executed, the medical support process proceeds to step ST50.

In a case where the controller 82C acquires the insertion step frame 40A from the second storage region 84B, the controller 82C may erase the insertion step frame 40A acquired from the second storage region 84B from the second storage region 84B. Moreover, in a case where all the insertion step frames 40A stored in the second storage region 84B are erased from the second storage region 84B, the controller 82C may issue a notification indicating that all the insertion step frames 40A stored in the second storage region 84B are erased from the second storage region 84B. Examples of the notification in this case include displaying information (for example, a text and/or a mark) for specifying that all the insertion step frames 40A stored in the second storage region 84B are erased from the second storage region 84B on the screen 35 or outputting a voice for specifying that all the insertion step frames 40A stored in the second storage region 84B are erased from the second storage region 84B from a speaker (not shown).

In step ST50, the controller 82C displays the insertion step frame 40A acquired from the second storage region 84B in the second display region 38. In a case where the insertion step frame 40A is already displayed in the second display region 38, the controller 82C updates the insertion step frame 40A to be displayed in the second display region 38 to the latest insertion step frame 40A acquired from the second storage region 84B. After the process in step ST50 is executed, the medical support process proceeds to step ST52.

In step ST52, the controller 82C determines whether or not a medical support process end condition is satisfied. An example of the medical support process end condition is a condition that an instruction for the endoscope apparatus 10 to end the medical support process is given (for example, a condition that the reception device 64 receives an instruction to end the medical support process).

In a case where the medical support process end condition is not satisfied in step ST52, a negative determination is made, and the medical support process proceeds to step ST10. In a case where the medical support process end condition is satisfied in step ST52, a positive determination is made, and the medical support process ends.

As described above, in the endoscope apparatus 10 according to the first embodiment, the first specifying process is executed based on the first movement distance 112A obtained in the insertion step 41A and the second movement distance 112B obtained in the removal step 41B. In the first embodiment, in a case where the first difference 124 and the second difference 126 match, the first specifying process is executed. The first difference 124 is a difference between the first reference movement distance 120 (that is, the movement distance 112 on the insertion path 29A (see FIG. 1) from the anus to the ileocecal portion 28A) and the first movement distance 112A, and the second difference 126 is a difference between the first reference movement distance 120 and the second movement distance 112B. The first specifying process is a process for specifying the identity between the lesion 42 shown in the insertion step frame 40A and the lesion 42 shown in the removal step frame 40B. Therefore, with the endoscope apparatus 10 according to the first embodiment, the doctor 12 can specify whether or not the same lesion 42 is imaged by the camera 52 in the insertion step 41A and the removal step 41B.

In addition, in the endoscope apparatus 10 according to the first embodiment, in a case where the lesion 42 is shown in the removal step frame 40B in the removal step 41B, the controller 82C determines whether or not the first difference 124 and the second difference 126 match. The first difference 124 corresponds to the distance from the lesion 42 found in the insertion step 41A to the ileocecal portion 28A, and the second difference 126 corresponds to the distance from the ileocecal portion 28A to the lesion 42 found in the removal step 41B. In a case where the first difference 124 and the second difference 126 match, the controller 82C determines that the level of the identity between the lesion 42 shown in the insertion step frame 40A and the lesion 42 shown in the removal step frame 40B is equal to or higher than a predetermined level. In a case where the controller 82C determines that the level of the identity between the lesion 42 shown in the insertion step frame 40A and the lesion 42 shown in the removal step frame 40B is equal to or higher than the predetermined level, the first display process 128 is executed by the controller 82C. In the first display process 128, the removal step frame 40B to which the second movement distance 112B used to calculate the second difference 126 matching the first difference 124 is added is displayed in the first display region 36, and the insertion step frame 40A to which the first movement distance 112A used to calculate the first difference 124 is added is displayed in the second display region 38.

Therefore, with the endoscope apparatus 10 according to the first embodiment, in a case where the controller 82C determines that the level of the identity between the lesion 42 shown in the insertion step frame 40A and the lesion 42 shown in the removal step frame 40B is equal to or higher than the predetermined level, the doctor 12 can visually ascertain an aspect of the lesion 42 shown in the removal step frame 40B displayed in the first display region 36 and an aspect of the lesion 42 shown in the insertion step frame 40A displayed in the second display region 38.

In addition, in the endoscope apparatus 10 according to the first embodiment, the insertion step tag 116 is added to the insertion step frame 40A, and the removal step tag 118 is added to the removal step frame 40B. The insertion step tag 116 is a tag for identifying that the insertion step frame 40A is the frame 40 obtained by imaging the region including the lesion 42 by the camera 52 in the insertion step 41A. The removal step tag 118 is a tag for identifying that the removal step frame 40B is the frame 40 obtained by imaging the region including the lesion 42 by the camera 52 in the removal step 41B.

Therefore, with the endoscope apparatus 10 according to the first embodiment, it is possible to easily identify that the insertion step frame 40A is the frame 40 obtained by imaging the region including the lesion 42 by the camera 52 in the insertion step 41A, and the removal step frame 40B is the frame 40 obtained by imaging the region including the lesion 42 by the camera 52 in the removal step 41B. As a result, it is easy to perform a process using the insertion step frame 40A and the removal step frame 40B.

In addition, in the endoscope apparatus 10 according to the first embodiment, the position of the camera 52 inside the large intestine 28 is defined based on the movement distance 112. The movement distance 112 is estimated by the movement estimation model 94 by inputting the plurality of frames 40 along the time series to the movement estimation model 94. Therefore, with the endoscope apparatus 10 according to the first embodiment, it is possible to estimate the position of the camera 52 inside the large intestine 28 without using a sensor specialized for detecting the position of the camera 52 inside the large intestine 28.

In the first embodiment, a form example has been described in which the insertion step frame 40A is displayed in the second display region 38 on a condition that the first difference 124 and the second difference 126 match, but the present disclosure is not limited to this. For example, the insertion step frame 40A may be displayed in the second display region 38 on a condition that the insertion step frame 40A to which the first movement distance 112A used to calculate the first difference 124 matching the second difference 126 is added is continuously obtained for a first predetermined number of frames (for example, a plurality of frames designated in advance) or more. In this case, the insertion step frame 40A displayed in the second display region 38 may be, among a plurality of the insertion step frames 40A continuously obtained for the first predetermined number of frames or more, the insertion step frame 40A obtained first in time, may be the insertion step frame 40A obtained last in time, or may be the insertion step frame 40A obtained between the insertion step frame 40A obtained first in time and the insertion step frame 40A obtained last in time. In addition, among the plurality of insertion step frames 40A continuously obtained for the first predetermined number of frames or more, the insertion step frame 40A in which a position at which the lesion 42 is shown is closest to a center of the angle of view of the camera 52 may be displayed in the second display region 38. In addition, all the plurality of insertion step frames 40A continuously obtained for the first predetermined number of frames or more or a plurality of the insertion step frames 40A selected according to some rule may be displayed in the second display region 38 in a comparable state.

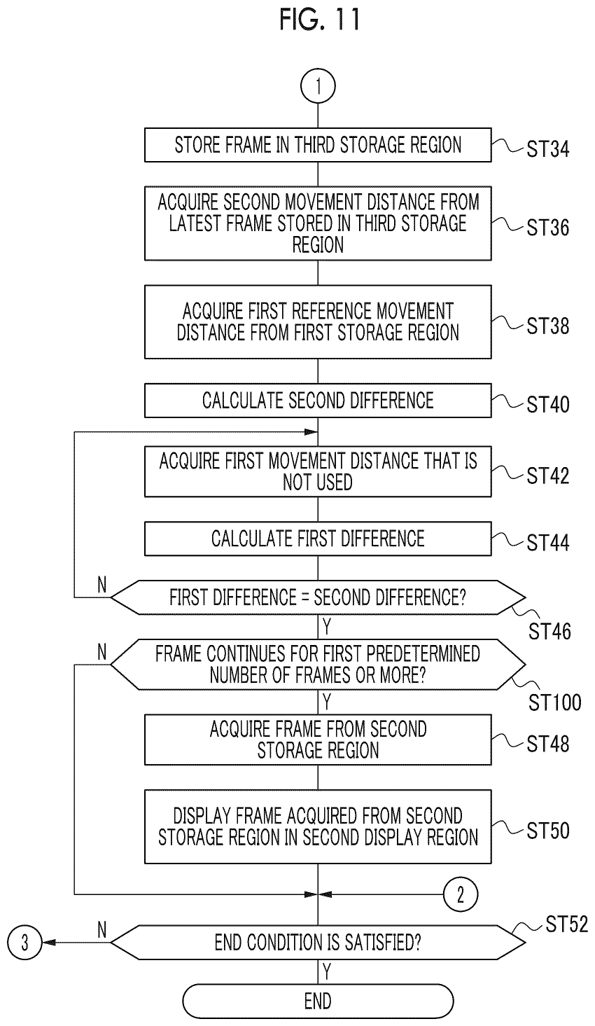

FIG. 11 shows an example of a flow of a medical support process for realizing this form example. The medical support process shown in FIG. 11 is different from the medical support process shown in FIG. 10B in that a process of step ST100 is provided between the process of step ST46 and the process of step ST48. In step ST100 shown in FIG. 11, the controller 82C determines whether or not the insertion step frame 40A to which the first movement distance 112A used to calculate the first difference 124 matching the second difference 126 is added continues for the first predetermined number of frames or more. In step ST100, in a case where the insertion step frame 40A to which the first movement distance 112A used to calculate the first difference 124 matching the second difference 126 is added does not continue for the first predetermined number of frames or more, a negative determination is made, and the medical support process proceeds to step ST52. In step ST100, in a case where the insertion step frame 40A to which the first movement distance 112A used to calculate the first difference 124 matching the second difference 126 is added continues for the first predetermined number of frames or more, a positive determination is made, and the medical support process proceeds to step ST48.

In this way, the insertion step frame 40A is displayed in the second display region 38 on a condition that the insertion step frame 40A to which the first movement distance 112A used to calculate the first difference 124 matching the second difference 126 is added is continuously obtained for the first predetermined number of frames or more, so that the doctor 12 can observe the lesion 42 (in other words, the lesion 42 recognized by the recognition unit 82A) that is highly likely to be observed in the insertion step 41A by the doctor 12 in the removal step 41B.

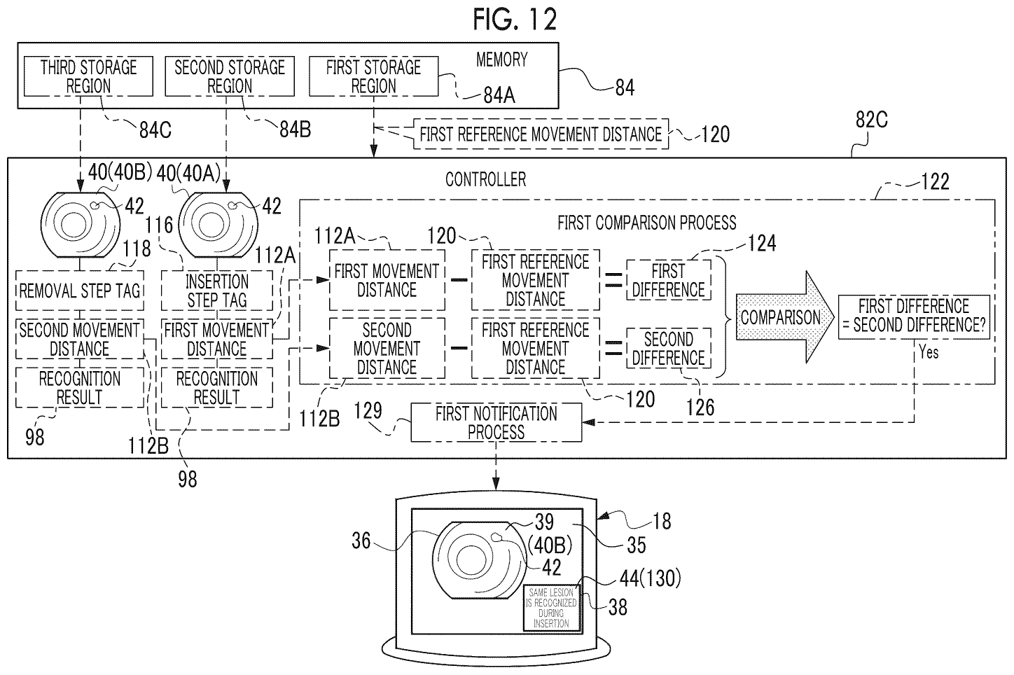

In the first embodiment, although a form example has been described in which the controller 82C executes the first display process 128, this is merely an example. For example, as shown in FIG. 12, the controller 82C may execute a first notification process 129. The first notification process 129 is a process of issuing a notification that the level of the identity between the lesion 42 shown in the insertion step frame 40A and the lesion 42 shown in the removal step frame 40B is equal to or higher than a predetermined level in a case where the level of the identity between the lesion 42 shown in the insertion step frame 40A and the lesion 42 shown in the removal step frame 40B is equal to or higher than the predetermined level.

Examples of the notification include an example in which a message 130 for notifying that the level of the identity between the lesion 42 shown in the insertion step frame 40A and the lesion 42 shown in the removal step frame 40B is equal to or higher than the predetermined level is displayed in the second display region 38 as one of pieces of the assistance information 44. In addition, a mark, a numerical value, or the like for notifying that the level of the identity between the lesion 42 shown in the insertion step frame 40A and the lesion 42 shown in the removal step frame 40B is equal to or higher than the predetermined level may be displayed in the second display region 38 as one of pieces of the assistance information 44. In addition, the insertion step frame 40A may also be displayed in the second display region 38 as in the first embodiment. In addition, information for giving a notification that the level of the identity between the lesion 42 shown in the insertion step frame 40A and the lesion 42 shown in the removal step frame 40B is equal to or higher than the predetermined level may be displayed in a region other than the second display region 38 in the screen 35 or may be displayed on a screen of a display device different from the display device 18. In addition, a voice for notifying that the level of the identity between the lesion 42 shown in the insertion step frame 40A and the lesion 42 shown in the removal step frame 40B is equal to or higher than the predetermined level may be output from a speaker (not shown).

In this way, by executing the first notification process 129 by the controller 82C, the doctor 12 can perceive that the level of the identity between the lesion 42 shown in the insertion step frame 40A and the lesion 42 shown in the removal step frame 40B is equal to or higher than the predetermined level.

### Second Embodiment

In the first embodiment, a form example has been described in which a process (for example, the first display process 128) for specifying the identity between the lesion 42 shown in the removal step frame 40B and the lesion 42 shown in the insertion step frame 40A obtained in the insertion step 41A is executed in a case where the removal step frame 40B is obtained in the removal step 41B. However, in the second embodiment, a form example will be described in which a process for specifying the identity between the lesion 42 shown in the frame 40 obtained in a step in which the camera 52 is pushed to the ileocecal portion 28A side (that is, a far side) inside the large intestine 28 and the lesion 42 shown in the frame 40 obtained in a step in which the camera 2 is pulled to the anal side (that is, a near side) from the inside of the large intestine 28 is performed. Here, the “near side” is synonymous with the entrance side of the large intestine 28.

In the second embodiment, the same constituents as those in the first embodiment are denoted by the same references, and the descriptions thereof will not be repeated. In addition, the above descriptions regarding FIGS. 11 and 12 (for example, form examples shown in FIGS. 11 and 12 and modification examples from the form examples shown in FIGS. 11 and 12) are also applicable to the second embodiment.

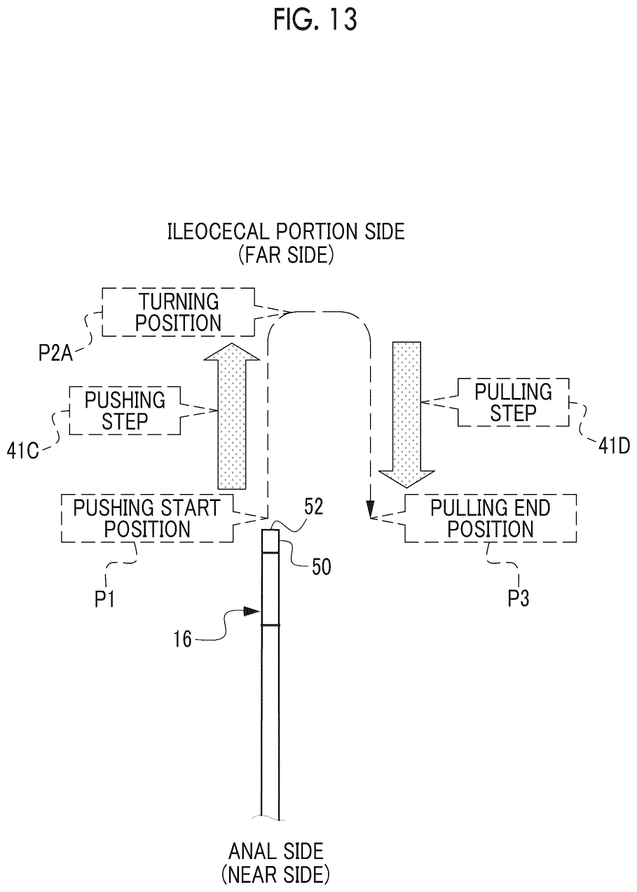

FIG. 13 is a conceptual diagram showing an example of an aspect in which a transition is made from a pushing step 41C that is a step in which the camera 52 is pushed into the far side (for example, the ileocecal portion 28A side) inside the large intestine 28 to a pulling step 41D that is a step in which the camera 52 is pulled from the inside of the large intestine 28 to the near side (for example, the anal side).

In the example shown in FIG. 13, the pushing step 41C is a step in which the camera 52 is moved from a pushing start position P1 which is a start position of the pushing step 41C to a turning position P2A which is a start position of the pulling step 41D (in other words, an end position of the pushing step 41C). In the example shown in FIG. 13, the pulling step 41D is a step in which the camera 52 is moved from the turning position P2A to a pulling end position P3 which is an end position of the pulling step 41D.

The turning position P2A is a position at which the camera 52 is stationary. For example, the turning position P2A is recognized based on a plurality of the movement distances 112 obtained by the controller 82C by estimation for each frame 40. That is, in a case where a difference between the plurality of movement distances 112 in the time series estimated over the plurality of frames 40 is within an allowable error determined in advance, the controller 82C determines that the camera 52 reaches the turning position P2A.

In the second embodiment, a direction in which the camera 52 advances in the pushing step 41C matches a direction in which the camera 52 advances in the insertion step 41A described in the first embodiment, and a direction in which the camera 52 advances in the pulling step 41D matches a direction in which the camera 52 advances in the removal step 41B described in the first embodiment. The pulling end position P3 in a case where a transition is made from the pushing step 41C to the pulling step 41D in the insertion step 41A is a position at which the camera 52 is stationary, and is recognized in the same manner as the turning position P2A is recognized.

In addition, in the second embodiment, a stroke in which the camera 52 moves in the pushing step 41C is shorter than a stroke (for example, the insertion path 29A shown in FIG. 1) in which the camera 52 moves in the insertion step 41A. In addition, in the second embodiment, a stroke in which the camera 52 moves in the pulling step 41D is shorter than a stroke (for example, the removal path 29B shown in FIG. 1) in which the camera 52 moves in the removal step 41B.

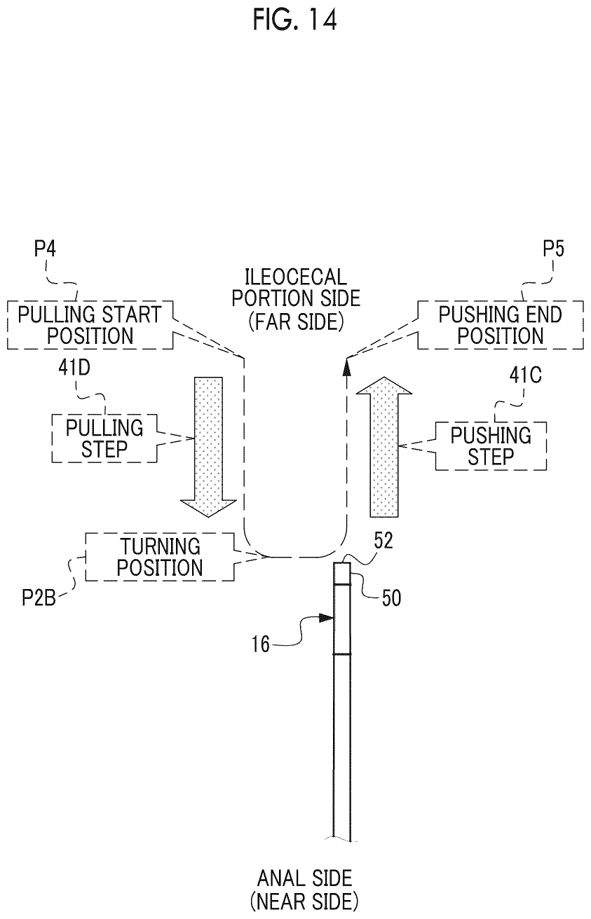

FIG. 14 is a conceptual diagram showing an example of an aspect in which a transition is made from the pulling step 41D to the pushing step 41C.

In the example shown in FIG. 14, the pulling step 41D is a step in which the camera 52 moves from a pulling start position P4 which is a start position of the pulling step 41D to a turning position P2B which is a start position of the pushing step 41C (in other words, an end position of the pulling step 41D). In addition, in the example shown in FIG. 14, the pushing step 41C is a step in which the camera 52 moves from the turning position P2B to a pushing end position P5 which is an end position of the pushing step 41C.

The turning position P2B is a position at which the camera 52 is stationary. For example, the turning position P2B is recognized based on a plurality of the movement distances 112 obtained by the controller 82C by estimation for each frame 40. That is, in a case where a difference between the plurality of movement distances 112 in the time series estimated over the plurality of frames 40 is within an allowable error determined in advance, the controller 82C determines that the camera 52 reaches the turning position P2B. The pushing end position P5 in a case where a transition is made from the pulling step 41D to the pushing step 41C in the removal step 41B is a position at which the camera 52 is stationary, and is recognized in the same manner as the turning position P2B is recognized.

Hereinafter, for convenience of description, in a case where the distinction is not necessary for description, the turning positions P2A and P2B are referred to as a “turning position P2”.

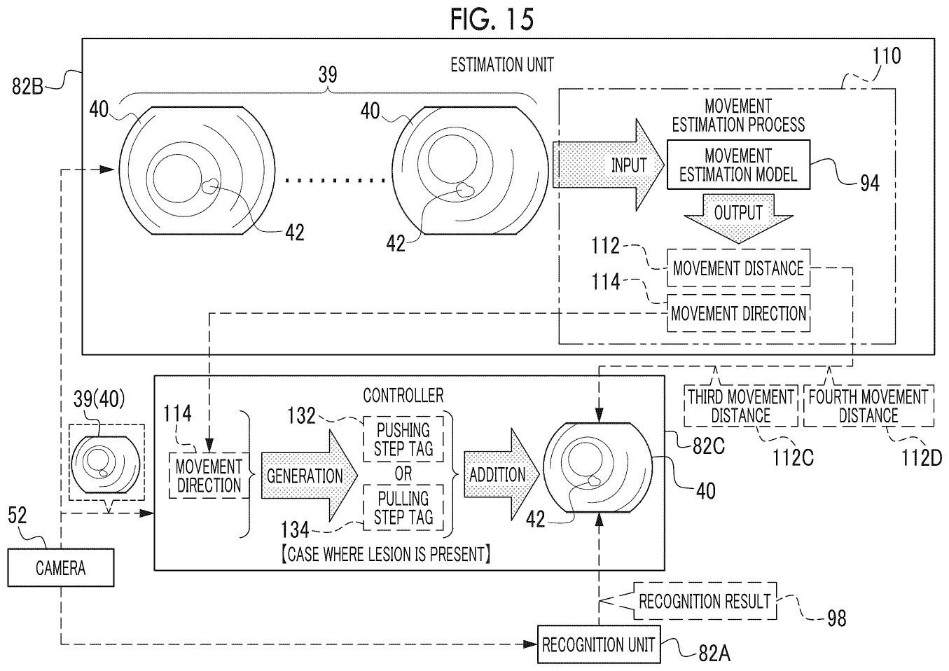

FIG. 15 is a conceptual diagram showing an example of processing contents of the estimation unit 82B and the controller 82C in the pushing step 41C and the pulling step 41D.

As shown in FIG. 15, the controller 82C acquires the recognition result 98 from the recognition unit 82A, and determines whether or not the lesion 42 is shown in the frame 40 input to the recognition model 92 by referring to the lesion presence/absence information 100 included in the acquired recognition result 98. Here, in a case where the lesion 42 is shown in the frame 40 that is input to the recognition model 92 in order to obtain the recognition result 98 acquired from the recognition unit 82A by the controller 82C, the controller 82C generates a pushing step tag 132 or a pulling step tag 134 based on the movement direction 114 input from the estimation unit 82B. The pushing step tag 132 refers to a tag that is an identifier for identifying the frame 40 obtained by imaging a region including the lesion 42 (that is, a region of a part of the entire region of the intestinal wall 32 that falls within an angle of view of the camera 52 and includes the lesion 42) by the camera 52 in the pushing step 41C. The pulling step tag 134 refers to a tag that is an identifier for identifying the frame 40 obtained by imaging a region including the lesion 42 (that is, a region of a part of the entire region of the intestinal wall 32 that falls within an angle of view of the camera 52 and includes the lesion 42) by the camera 52 in the pulling step 41D.

In a case where the controller 82C generates the pushing step tag 132 or the pulling step tag 134 in this way, first, the controller 82C determines whether the movement direction 114 input from the estimation unit 82B is a pushing direction or a pulling direction. Moreover, in a case where the movement direction 114 input from the estimation unit 82B is the pushing direction, the controller 82C generates the pushing step tag 132, and in a case where the movement direction 114 input from the estimation unit 82B is the pulling direction, the controller 82C generates the pulling step tag 134. In addition, in the second embodiment, the pushing step tag 132 is an example of a “pushing step identifier” according to the present disclosure, and the pulling step tag 134 is an example of a “pulling step identifier” according to the present disclosure.

The controller 82C adds the pushing step tag 132 or the pulling step tag 134, the movement distance 112, and the recognition result 98 to the frame 40 which is acquired from the camera 52 and in which the lesion 42 is shown. The frame 40 to which the pushing step tag 132 or the pulling step tag 134, the movement distance 112, and the recognition result 98 are added is the same frame 40 as the latest frame 40 input to the movement estimation model 94 for estimating the movement direction 114 used to generate the pushing step tag 132 or the pulling step tag 134. The movement distance 112 added to the frame 40 is the movement distance 112 estimated by the movement estimation model 94 together with the movement direction 114 used to generate the pushing step tag 132 or the pulling step tag 134. The recognition result 98 added to the frame 40 is the recognition result 98 used by the controller 82C to determine whether or not the lesion 42 is present.

The movement distance 112 estimated by the movement estimation model 94 together with the movement direction 114 used to generate the pushing step tag 132 is added to the frame 40 as a third movement distance 112C. The movement distance 112 estimated by the movement estimation model 94 together with the movement direction 114 used to generate the pulling step tag 134 is added to the frame 40 as a fourth movement distance 112D.

Here, the third movement distance 112C is an example of “pushing position specifying information” according to the present disclosure, and the fourth movement distance 112D is an example of “pulling position specifying information” according to the present disclosure.

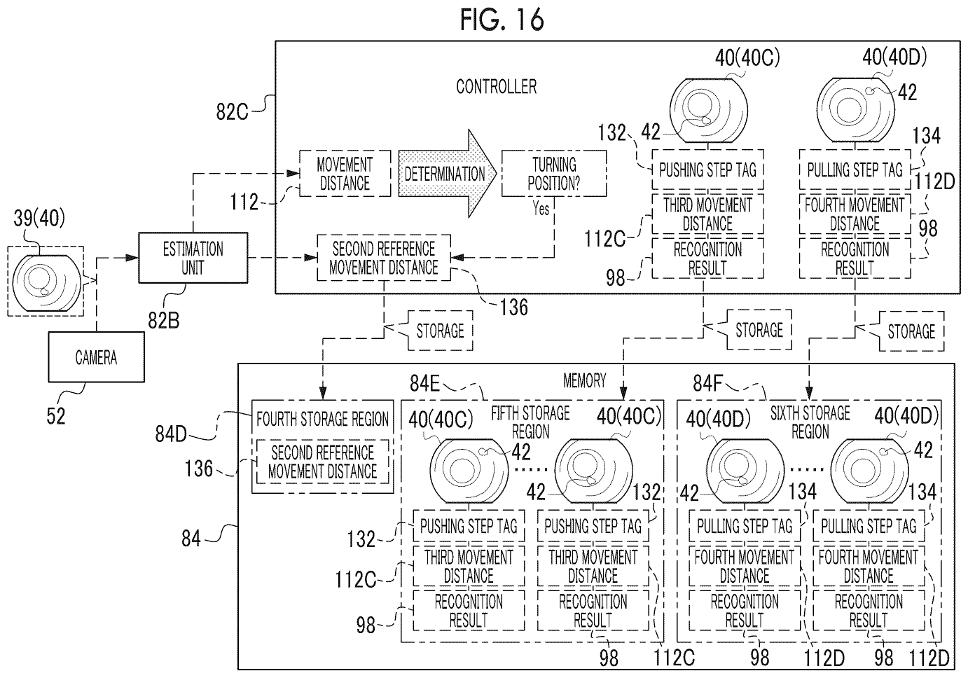

FIG. 16 is a conceptual diagram showing an example of an aspect in which the controller 82C stores various kinds of information in the memory 84. As shown in FIG. 16, the memory 84 includes a fourth storage region 84D, a fifth storage region 84E, and a sixth storage region 84F. The controller 82C stores a pushing step frame 40C in the fifth storage region 84E in time series. The pushing step frame 40C refers to the frame 40 obtained by imaging the region including the lesion 42 in the large intestine 28 by the camera 52 in the pushing step 41C (in the example shown in FIG. 16, the frame 40 to which the pushing step tag 132, the third movement distance 112C, and the recognition result 98 are added). In addition, the controller 82C stores a pulling step frame 40D in the sixth storage region 84F in time series. The pulling step frame 40D refers to the frame 40 obtained by imaging the region including the lesion 42 in the large intestine 28 by the camera 52 in the pulling step 41D (in the example shown in FIG. 16, the frame 40 to which the pulling step tag 134, the fourth movement distance 112D, and the recognition result 98 are added). Here, the pushing step frame 40C is an example of a “pushing step frame” according to the present disclosure, and the pulling step frame 40D is an example of a “pulling step frame” according to the present disclosure.

The controller 82C acquires a plurality of the movement distances 112 from the estimation unit 82B, and determines whether or not the camera 52 has reached the turning position P2 based on the plurality of movement distances 112 (for example, the plurality of movement distances 112 continuously obtained in time series). That is, in a case where a difference between the plurality of movement distances 112 acquired from the estimation unit 82B is within an allowable error determined in advance, the controller 82C determines that the camera 52 has reached the turning position P2.

In a case where the camera 52 has reached the turning position P2, the controller 82C acquires the movement distance 112 estimated by the estimation unit 82B from the estimation unit 82B as a second reference movement distance 136. The controller 82C stores the second reference movement distance 136 in the fourth storage region 84D.

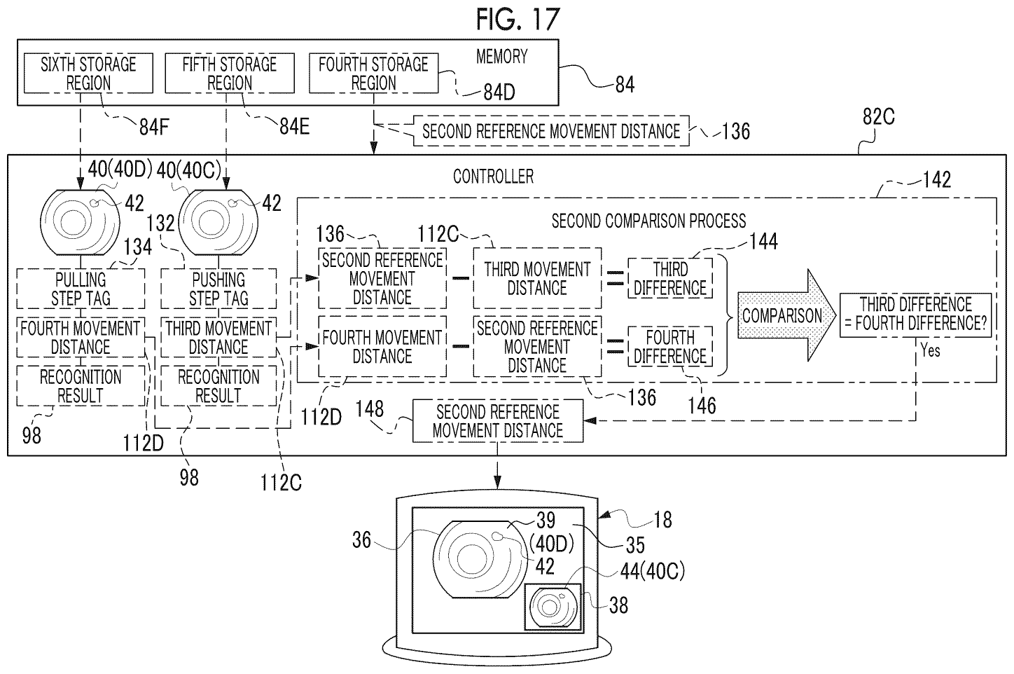

FIG. 17 is a conceptual diagram showing an example of processing contents performed by the controller 82C based on the second reference movement distance 136 stored in the fourth storage region 84D, the third movement distance 112C added to the pushing step frame 40C stored in the fifth storage region 84E, and the fourth movement distance 112D added to the pulling step frame 40D stored in the sixth storage region 84F in a case where a transition is made from the pushing step 41C to the pulling step 41D in the insertion step 41A or the removal step 41B.

As shown in FIG. 17, the controller 82C acquires the second reference movement distance 136 from the fourth storage region 84D. In addition, the controller 82C acquires the third movement distance 112C added to the pushing step frame 40C from the fifth storage region 84E. Further, the controller 82C acquires the fourth movement distance 112D added to the latest pulling step frame 40D from the sixth storage region 84F. Then, the controller 82C performs a second comparison process 142 based on the second reference movement distance 136, the third movement distance 112C, and the fourth movement distance 112D.

In the second comparison process 142, a third difference 144 is calculated. The third difference 144 refers to a difference between the second reference movement distance 136 and the third movement distance 112C. The third difference 144 is calculated for each third movement distance 112C added to each of all the pushing step frames 40C stored in the fifth storage region 84E. In addition, here, as an example of the difference between the second reference movement distance 136 and the third movement distance 112C, a value obtained by subtracting the third movement distance 112C from the second reference movement distance 136 is used.

In the second comparison process 142, a fourth difference 146 is calculated. The fourth difference 146 refers to a difference between the fourth movement distance 112D added to the latest pulling step frame 40D stored in the sixth storage region 84F and the second reference movement distance 136. Here, as an example of the difference between the fourth movement distance 112D and the second reference movement distance 136, a value obtained by subtracting the second reference movement distance 136 from the fourth movement distance 112D is used.

In the example shown in FIG. 17, a form example has been described in which the value obtained by subtracting the third movement distance 112C from the second reference movement distance 136 is used as the third difference 144, and the value obtained by subtracting the second reference movement distance 136 from the fourth movement distance 112D is used as the fourth difference 146. However, this is merely an example. For example, the third difference 144 may be an absolute value of the difference between the second reference movement distance 136 and the third movement distance 112C, and the fourth difference 146 may be an absolute value of the difference between the second reference movement distance 136 and the fourth movement distance 112D. In addition, the third difference 144 may be a value obtained by subtracting the second reference movement distance 136 from the third movement distance 112C, and the fourth difference 146 may be a value obtained by subtracting the fourth movement distance 112D from the second reference movement distance 136.

In the second comparison process 142, it is determined whether or not each third difference 144 calculated for each third movement distance 112C added to each of all the pushing step frames 40C stored in the fifth storage region 84E matches the fourth difference 146. Here, the term “match” means match including an error that is generally allowed in the technical field to which the present disclosure belongs and does not deviate from the gist of the present disclosure, in addition to perfect match.

In a case where it is determined that the third difference 144 and the fourth difference 146 match, the controller 82C executes a second specifying process for specifying an identity between the lesion 42 shown in the pushing step frame 40C to which the third movement distance 112C used to calculate the third difference 144 is added and the lesion 42 shown in the pulling step frame 40D to which the fourth movement distance 112D used to calculate the fourth difference 146 that matches the third difference 144 is added. In the example shown in FIG. 17, a second display process 148 is shown as an example of a process included in the second specifying process. The second display process 148 is an example of a “second specifying process” and a “second display process” according to the present disclosure.

The second display process 148 is a process of displaying the pushing step frame 40C and the pulling step frame 40D on the screen 35 in a comparable state in a case where a level of the identity between the lesion 42 shown in the pushing step frame 40C and the lesion 42 shown in the pulling step frame 40D is equal to or higher than a predetermined level. Here, the predetermined level is an example of a “third predetermined level” and a “fourth predetermined level” according to the present disclosure. In the second embodiment, a case where the level of the identity between the lesion 42 shown in the pushing step frame 40C and the lesion 42 shown in the pulling step frame 40D is equal to or higher than the predetermined level indicates a case where the third difference 144 and the fourth difference 146 match each other.

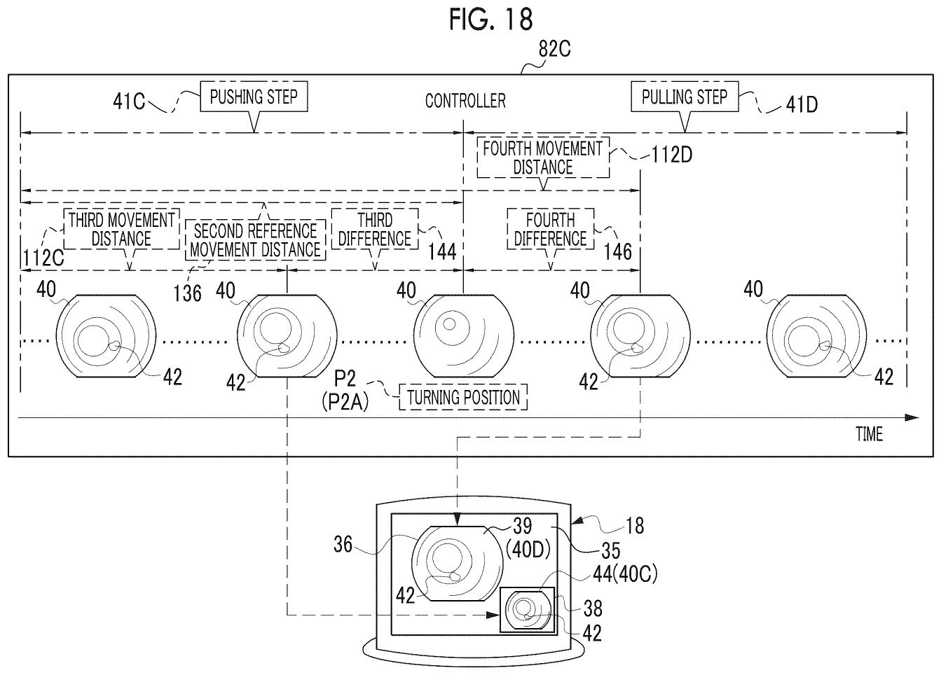

For example, as shown in FIG. 18, in a case where the third difference 144 corresponding to a distance from the lesion 42 found in the pushing step 41C to the turning position P2 (in the example shown in FIG. 18, the turning position P2A) and the fourth difference 146 corresponding to a distance from the turning position P2 to the lesion 42 found in the pulling step 41D match, the controller 82C determines that the level of the identity between the lesion 42 shown in the pushing step frame 40C and the lesion 42 shown in the pulling step frame 40D is equal to or higher than the predetermined level.

As shown in FIGS. 17 and 18, in the second display process 148, the pulling step frame 40D to which the fourth movement distance 112D used to calculate the fourth difference 146 matching the third difference 144 is added is displayed in the first display region 36, and the pushing step frame 40C to which the third movement distance 112C used to calculate the third difference 144 is added is displayed in the second display region 38. As a result, the pulling step frame 40D displayed in the first display region 36 and the pushing step frame 40C displayed in the second display region 38 are in a visually comparable state. In addition, the lesion 42 can be visually specified from the pulling step frame 40D displayed in the first display region 36, and the lesion 42 can be visually specified from the pushing step frame 40C displayed in the second display region 38. Therefore, the lesion 42 shown in the pulling step frame 40D displayed in the first display region 36 and the lesion 42 shown in the pushing step frame 40C displayed in the second display region 38 are in a visually comparable state. Here, the pulling step frame 40D displayed in the first display region 36 is an example of a “fourth image” according to the present disclosure, and the pushing step frame 40C displayed in the second display region 38 is an example of a “third image” according to the present disclosure.

In the examples shown in FIGS. 17 and 18, a from example has been described for a case where a transition is made from the pushing step 41C to the pulling step 41D (that is, a case where the pushing step 41C precedes the pulling step 41D). However, the process is performed in the same manner also for a case where a transition is made from the pulling step 41D to the pushing step 41C (that is, a case where the pulling step 41D precedes the pushing step 41C). In this case, for example, the pushing step frame 40C is displayed in the first display region 36, and the pulling step frame 40D is displayed in the second display region 38. As a result, the lesion 42 shown in the pushing step frame 40C displayed in the first display region 36 and the lesion 42 shown in the pulling step frame 40D displayed in the second display region 38 are in a visually comparable state.

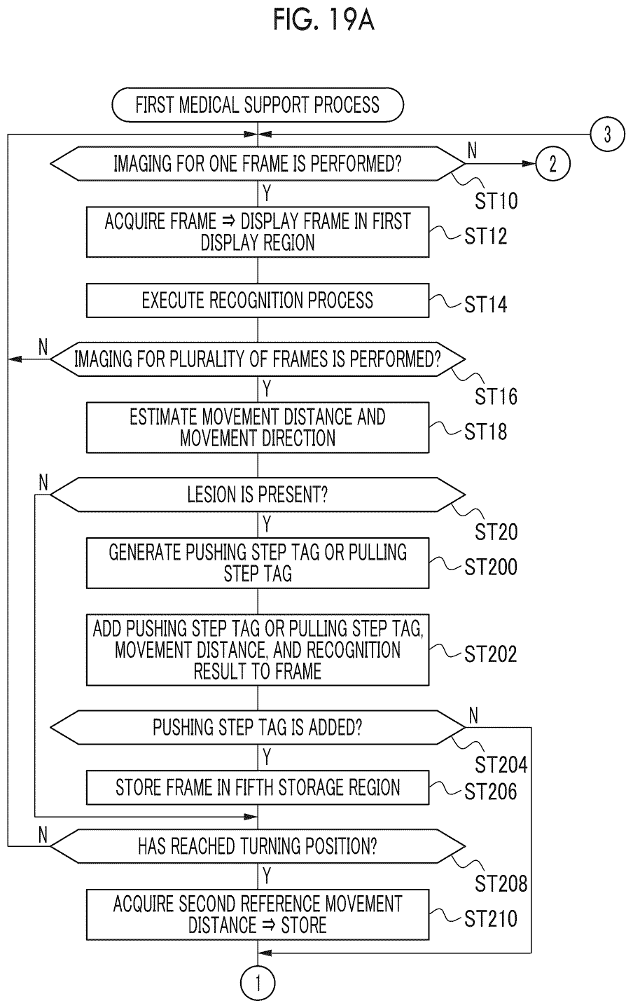

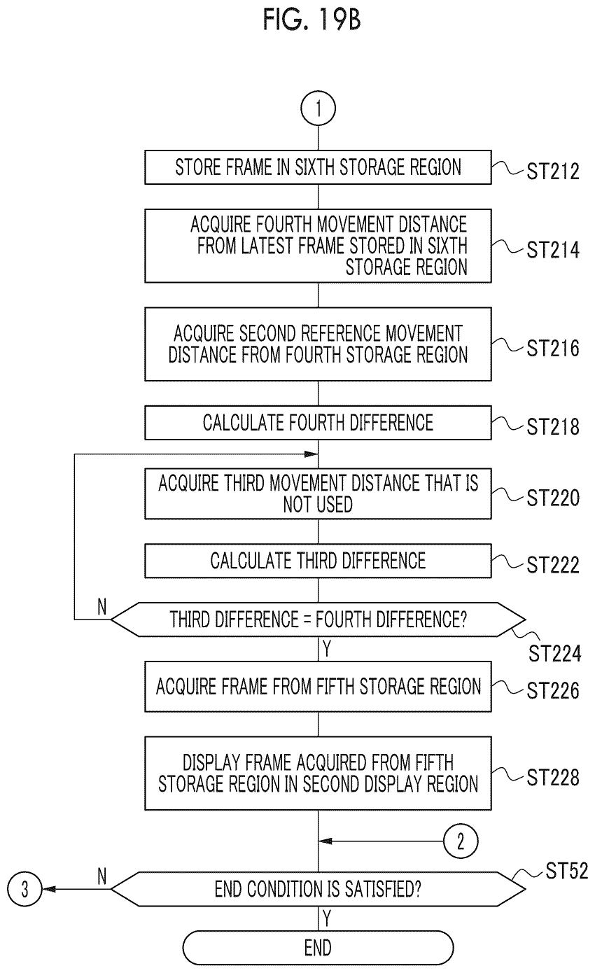

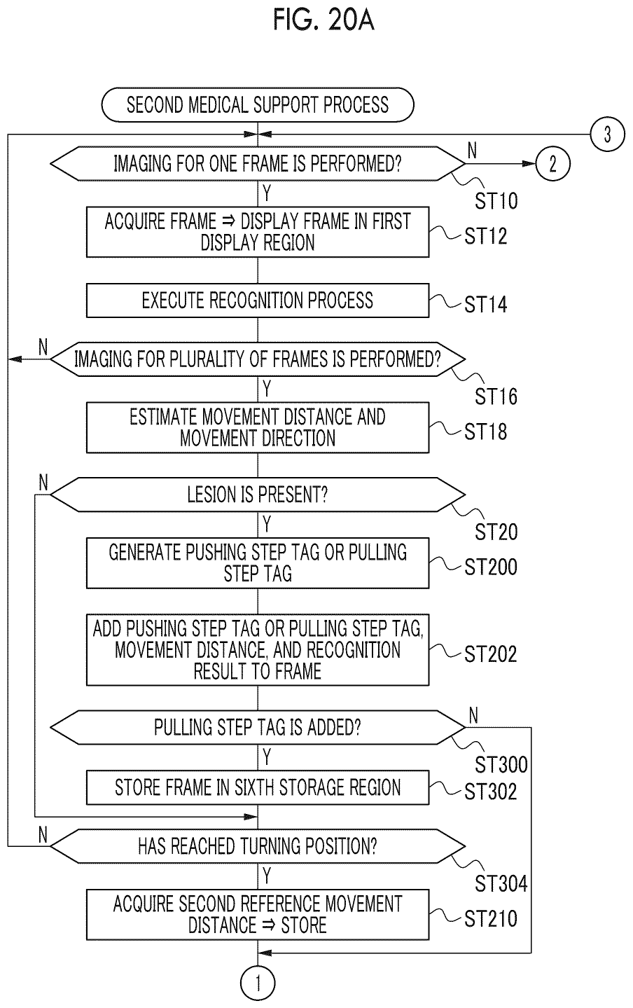

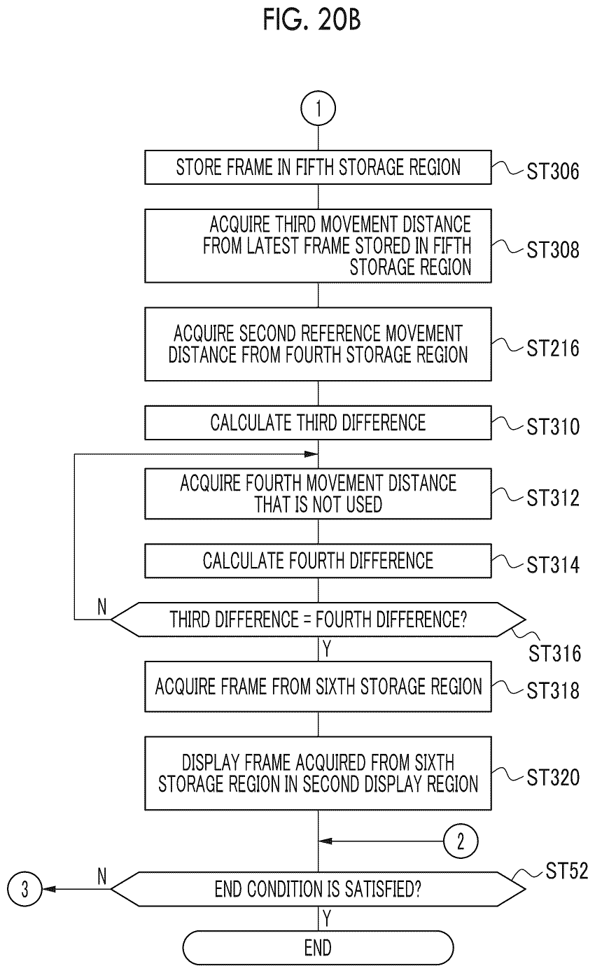

Next, an operation of a part of the endoscope apparatus 10 according to the second embodiment of the present disclosure will be described with reference to FIGS. 19A, 19B, 20A, and 20B.

FIGS. 19A, 19B, 20A, and 20B show an example of a flow of the medical support process according to the second embodiment. The medical support process according to the second embodiment is classified into a first medical support process and a second medical support process. FIGS. 19A and 19B show an example of a flow of the first medical support process.

FIGS. 20A and 20B show an example of a flow of the second medical support process. The first medical support process and the second medical support process may be selectively executed by the processor 82 or may be executed in parallel by the processor 82. The flow of the medical support process according to the second embodiment shown in FIGS. 19A, 19B, 20A, and 20B is an example of a “medical support method” according to the present disclosure.

In the second embodiment, the processor 82 may execute the medical support process according to the second embodiment in parallel with the medical support process according to the first embodiment, or may execute the medical support process according to the second embodiment without executing the medical support process according to the first embodiment. In a case where the medical support process according to the second embodiment is executed in parallel with the medical support process according to the first embodiment, for example, a screen on which a processing result of the medical support process according to the first embodiment is displayed and a screen on which a processing result of the medical support process according to the second embodiment is displayed may be separately provided, or the processing result of the medical support process according to the first embodiment and the processing result of the medical support process according to the second embodiment may be distinguishably displayed in a plurality of display regions provided in one screen (for example, the screen 35).

Here, first, the first medical support process will be described with reference to FIGS. 19A and 19B. The first medical support process is an example of a process assuming a case where a transition is made from the pushing step 41C to the pulling step 41D in the insertion step 41A or the removal step 41B (see FIG. 13), and is executed by the processor 82.

The flowcharts shown in FIGS. 19A and 19B are different from the flowcharts shown in FIGS. 10A and 10B in that steps ST200 to ST228 are provided instead of steps ST22 to ST50. In the following, in the flowcharts shown in FIGS. 19A and 19B, steps that are the same as the steps included in the flowcharts shown in FIGS. 10A and 10B are denoted by the same step numbers as the step numbers shown in FIGS. 10A and 10B, and the descriptions thereof will be omitted.

In addition, in the following, the description will be made on the premise that the inside of the large intestine 28 is irradiated with the same type of light (for example, light for BLI and/or light for LCI) in the pushing step 41C and the pulling step 41D, and a region (for example, the intestinal wall 32) irradiated with the light is imaged by the camera 52.

In the first medical support process shown in FIG. 19A, in step ST200, the controller 82C generates the pushing step tag 132 or the pulling step tag 134 based on the movement direction 114 estimated by the estimation unit 82B in step ST18. That is, in a case where the movement direction 114 is the pushing direction, the pushing step tag 132 is generated, and in a case where the movement direction 114 is the pulling direction, the pulling step tag 134 is generated. After the process in step ST200 is executed, the first medical support process proceeds to step ST204.

In step ST202, the controller 82C adds the pushing step tag 132 or the pulling step tag 134 generated in step ST200, the movement distance 112 estimated in step ST18, and the recognition result 98 obtained by executing the recognition process 96 in step ST14 to the frame 40 acquired in step ST12. For example, in a case where the movement direction 114 estimated in step ST18 is the pushing direction, the controller 82C adds the pushing step tag 132, adds the movement distance 112 estimated in step ST18 as the third movement distance 112C, and adds the recognition result 98, to the frame 40 acquired in step ST12, that is, the pushing step frame 40C. In addition, for example, in a case where the movement direction 114 estimated in step ST18 is the pulling direction, the controller 82C adds the pulling step tag 134, adds the movement distance 112 estimated in step ST18 as the fourth movement distance 112D, and adds the recognition result 98, to the frame 40 acquired in step ST12, that is, the pulling step frame 40D. After the process in step ST202 is executed, the first medical support process proceeds to step ST204.

In step ST204, the controller 82C determines whether or not the pushing step tag 132 is added to the frame 40. In step ST204, in a case where the pushing step tag 132 is not added to the frame 40 (that is, in a case where the pulling step tag 134 is added to the frame 40), a negative determination is made, and the first medical support process proceeds to step ST212 shown in FIG. 19B. In step ST204, in a case where the pushing step tag 132 is added to the frame 40, a positive determination is made, and the first medical support process proceeds to step ST206.

In step ST206, the controller 82C stores the pushing step frame 40C to which the pushing step tag 132, the movement distance 112 as the third movement distance 112C, and the recognition result 98 are added, in the fifth storage region 84E in time series. After the process in step ST206 is executed, the first medical support process proceeds to step ST208.

In step ST208, the controller 82C determines whether or not the camera 52 has reached the turning position P2A (refer to FIG. 13) by referring to the movement distance 112 for a plurality of frames estimated in step ST18 (that is, the movement distance 112 for a plurality of frames along the time series). In step ST208, in a case where the camera 52 has not reached the turning position P2A, a negative determination is made, and the first medical support process proceeds to step ST10. In step ST208, in a case where the camera 52 has reached the turning position P2A, a positive determination is made, and the first medical support process proceeds to step ST210.

In step ST210, the controller 82C stores the movement distance 112 estimated in step ST18 in the fourth storage region 84D as the second reference movement distance 136. After the process in step ST210 is executed, the first medical support process proceeds to step ST212 shown in FIG. 19B.

In step ST212 shown in FIG. 19B, the controller 82C stores the pulling step frame 40D to which the pulling step tag 134, the movement distance 112 as the fourth movement distance 112D, and the recognition result 98 are added, in the sixth storage region 84F in time series. After the process in step ST212 is executed, the first medical support process proceeds to step ST214.

In step ST214, the controller 82C acquires the fourth movement distance 112D from the latest pulling step frame 40D stored in the sixth storage region 84F. After the process in step ST214 is executed, the first medical support process proceeds to step ST216.

In step ST216, the controller 82C acquires the second reference movement distance 136 from the fourth storage region 84D. After the process in step ST216 is executed, the first medical support process proceeds to step ST218.

In step ST218, the controller 82C calculates the difference between the fourth movement distance 112D acquired in step 214 and the second reference movement distance 136 acquired in step ST216, that is, the fourth difference 146. After the process in step ST218 is executed, the first medical support process proceeds to step ST220.

In step ST220, the controller 82C acquires the third movement distance 112C that is not used in step ST220. After the process in step ST220 is executed, the first medical support process proceeds to step ST222.

In step ST222, the controller 82C calculates the difference between the third movement distance 112C acquired in step ST220 and the second reference movement distance 136 acquired in step ST216, that is, the third difference 144. After the process in step ST222 is executed, the first medical support process proceeds to step ST224.

In step ST224, the controller 82C determines whether or not the third difference 144 calculated in step ST222 and the fourth difference 146 calculated in step ST218 match. In step ST224, in a case where the third difference 144 and the fourth difference 146 do not match, a negative determination is made, and the first medical support process proceeds to step ST220. In step ST224, in a case where the third difference 144 and the fourth difference 146 match, a positive determination is made, and the first medical support process proceeds to step ST226.

In step ST226, the controller 82C acquires the pushing step frame 40C to which the third movement distance 112C used to calculate the third difference 144 matching the fourth difference 146 is added, from the fifth storage region 84E. After the process in step ST226 is executed, the first medical support process proceeds to step ST228.

In a case where the controller 82C acquires the pushing step frame 40C from the fifth storage region 84E, the controller 82C may erase the pushing step frame 40C acquired from the fifth storage region 84E from the fifth storage region 84E. Moreover, in a case where all the pushing step frames 40C stored in the fifth storage region 84E are erased from the fifth storage region 84E, the controller 82C may issue a notification indicating that all the pushing step frames 40C stored in the fifth storage region 84E are erased from the fifth storage region 84E. Examples of the notification in this case include displaying information (for example, a text and/or a mark) for specifying that all the pushing step frames 40C stored in the fifth storage region 84E are erased from the fifth storage region 84E on the screen 35 or outputting a voice for specifying that all the pushing step frames 40C stored in the fifth storage region 84E are erased from the fifth storage region 84E from a speaker (not shown).

In step ST228, the controller 82C displays the pushing step frame 40C acquired from the fifth storage region 84E in the second display region 38. In a case where the pushing step frame 40C is already displayed in the second display region 38, the controller 82C updates the pushing step frame 40C to be displayed in the second display region 38 to the latest pushing step frame 40C acquired from the fifth storage region 84E. After the process in step ST228 is executed, the first medical support process proceeds to step ST52.

Next, the second medical support process will be described with reference to FIGS. 20A and 20B. The second medical support process is an example of a process assuming a case where a transition is made from the pulling step 41D to the pushing step 41C in the insertion step 41A or the removal step 41B (see FIG. 14), and is executed by the processor 82.

Flowcharts shown in FIGS. 20A and 20B are different from the flowcharts shown in FIGS. 19A and 19B in that steps ST300 to ST304 are provided instead of steps ST204 to ST208, steps ST306 and ST308 are provided instead of steps ST212 and ST214, and steps ST310 to ST320 are provided instead of steps ST218 to ST228. In the following, in the flowcharts shown in FIGS. 20A and 20B, steps that are the same as the steps included in the flowcharts shown in FIGS. 10A, 10B, 19A, and 19B are denoted by the same step numbers as the step numbers shown in FIGS. 10A, 10B, 19A, and 19B, and the descriptions thereof will be omitted.

In addition, in a case where the first medical support process shown in FIGS. 19A and 19B and the second medical support process shown in FIGS. 20A and 20B are executed in parallel, the process of overlapping steps between the first medical support process shown in FIGS. 19A and 19B and the second medical support process shown in FIGS. 20A and 20B need only be executed in any one of the first medical support process or the second medical support process. In addition, in a case where the first medical support process shown in FIGS. 19A and 19B and the second medical support process shown in FIGS. 20A and 20B are executed in parallel, a screen on which a processing result of the first medical support process shown in FIGS. 19A and 19B is displayed and a screen on which a processing result of the second medical support process shown in FIGS. 20A and 20B is displayed may be separately provided, or the processing result of the first medical support process and the processing result of the second medical support process may be distinguishably displayed in a plurality of display regions provided in one screen (for example, the screen 35).

In addition, in a case where the medical support process according to the first embodiment, the first medical support process shown in FIGS. 19A and 19B, and the second medical support process shown in FIGS. 20A and 20B are executed in parallel, a screen on which the processing result of the medical support process according to the first embodiment is displayed, a screen on which the processing result of the first medical support process shown in FIGS. 19A and 19B is displayed, and a screen on which the processing result of the second medical support process shown in FIGS. 20A and 20B is displayed may be separately provided, or the processing result of the medical support process according to the first embodiment, the processing result of the first medical support process shown in FIGS. 19A and 19B, and the processing result of the second medical support process shown in FIGS. 20A and 20B may be distinguishably displayed in a plurality of display regions provided in one screen (for example, the screen 35).

In the second medical support process shown in FIG. 20A, in step ST300, the controller 82C determines whether or not the pulling step tag 134 is added to the frame 40. In step ST300, in a case where the pulling step tag 134 is not added to the frame 40 (that is, in a case where the pushing step tag 132 is attached to the frame 40), a negative determination is made, and the second medical support process proceeds to step ST306 shown in FIG. 20B. In step ST300, in a case where the pulling step tag 134 is added to the frame 40, a positive determination is made, and the second medical support process proceeds to step ST302.

In step ST302, the controller 82C stores the pulling step frame 40D to which the pulling step tag 134, the movement distance 112 as the fourth movement distance 112D, and the recognition result 98 are added, in the sixth storage region 84F in time series. After the process in step ST302 is executed, the second medical support process proceeds to step ST304.

In step ST304, the controller 82C determines whether or not the camera 52 has reached the turning position P2B (see FIG. 14) by referring to the movement distance 112 for a plurality of frames estimated in step ST18 (that is, the movement distance 112 for a plurality of frames along the time series). In step ST304, in a case where the camera 52 has not reached the turning position P2B, a negative determination is made, and the second medical support process proceeds to step ST10. In step ST304, in a case where the camera 52 has reached the turning position P2B, a positive determination is made, and the second medical support process proceeds to step ST210.

In step ST306 shown in FIG. 20B, the controller 82C stores the pushing step frame 40C to which the pushing step tag 132, the movement distance 112 as the third movement distance 112C, and the recognition result 98 are added, in the fifth storage region 84E in time series. After the process in step ST306 is executed, the second medical support process proceeds to step ST308.

In step ST308, the controller 82C acquires the third movement distance 112C from the latest pushing step frame 40C stored in the fifth storage region 84E. After the process in step ST308 is executed, the second medical support process proceeds to step ST216.

In step ST310, the controller 82C calculates the difference between the third movement distance 112C acquired in step 308 and the second reference movement distance 136 acquired in step ST216, that is, the third difference 144. After the process in step ST310 is executed, the second medical support process proceeds to step ST312.

In step ST312, the controller 82C acquires the fourth movement distance 112D that is not used in step ST312. After the process in step ST312 is executed, the second medical support process proceeds to step ST314.

In step ST314, the controller 82C calculates the difference between the fourth movement distance 112D acquired in step ST312 and the second reference movement distance 136 acquired in step ST216, that is, the fourth difference 146. After the process in step ST314 is executed, the second medical support process proceeds to step ST316.

In step ST316, the controller 82C determines whether or not the third difference 144 calculated in step ST310 and the fourth difference 146 calculated in step ST314 match. In step ST316, in a case where the third difference 144 and the fourth difference 146 do not match, a negative determination is made, and the second medical support process proceeds to step ST312. In step ST316, in a case where the third difference 144 and the fourth difference 146 match, a positive determination is made, and the second medical support process proceeds to step ST318.

In step ST318, the controller 82C acquires the pulling step frame 40D to which the fourth movement distance 112D used to calculate the fourth difference 146 matching the third difference 144 is added, from the sixth storage region 84F. After the process in step ST318 is executed, the second medical support process proceeds to step ST320.

In a case where the controller 82C acquires the pulling step frame 40D in the sixth storage region 84F, the controller 82C may erase the pulling step frame 40D acquired from the sixth storage region 84F from the sixth storage region 84F. Moreover, in a case where all the pulling step frames 40D stored in the sixth storage region 84F are erased from the sixth storage region 84F, the controller 82C may issue a notification indicating that all the pulling step frames 40D stored in the sixth storage region 84F are erased from the sixth storage region 84F. Examples of the notification in this case include displaying information (for example, a text and/or a mark) for specifying that all the pulling step frames 40D stored in the sixth storage region 84F are erased from the sixth storage region 84F on the screen 35 or outputting a voice for specifying that all the pulling step frames 40D stored in the sixth storage region 84F are erased from the sixth storage region 84F from a speaker (not shown).

In step ST320, the controller 82C displays the pulling step frame 40D acquired from the sixth storage region 84F in the second display region 38. In a case where the pulling step frame 40D is already displayed in the second display region 38, the controller 82C updates the pulling step frame 40D to be displayed in the second display region 38 to the latest pulling step frame 40D acquired from the sixth storage region 84F. After the process in step ST320 is executed, the second medical support process proceeds to step ST52.

As described above, in the endoscope apparatus 10 according to the second embodiment, the second specifying process is executed based on the third movement distance 112C obtained in the pushing step 41C and the fourth movement distance 112D obtained in the pulling step 41D. In the second embodiment, in a case where the third difference 144 and the fourth difference 146 match, the second specifying process is executed. The third difference 144 is a difference between the second reference movement distance 136 (that is, the movement distance 112 from the anus to the turning position P2) and the third movement distance 112C, and the fourth difference 146 is a difference between the second reference movement distance 136 and the fourth movement distance 112D. The second specifying process is a process for specifying the identity between the lesion 42 shown in the pushing step frame 40C and the lesion 42 shown in the pulling step frame 40D. Therefore, with the endoscope apparatus 10 according to the second embodiment, it is possible to specify whether or not the same lesion 42 is imaged by the camera 52 in the pushing step 41C and the pulling step 41D.

In addition, in the endoscope apparatus 10 according to the second embodiment, in a case where the pushing step 41C precedes the pulling step 41D (see FIG. 13), and the lesion 42 is shown in the pulling step frame 40D in the pulling step 41D, the controller 82C determines whether or not the third difference 144 and the fourth difference 146 match. The third difference 144 corresponds to a distance from the lesion 42 found in the pushing step 41C to the turning position P2A (see FIG. 13), and the fourth difference 146 corresponds to a distance from the turning position P2A to the lesion 42 found in the pulling step 41D. In a case where the third difference 144 and the fourth difference 146 match, the controller 82C determines that the level of the identity between the lesion 42 shown in the pushing step frame 40C and the lesion 42 shown in the pulling step frame 40D is equal to or higher than a predetermined level. In a case where the controller 82C determines that the level of the identity between the lesion 42 shown in the pushing step frame 40C and the lesion 42 shown in the pulling step frame 40D is equal to or higher than the predetermined level, the second display process 148 is executed by the controller 82C. In the second display process 148, the pulling step frame 40D to which the fourth movement distance 112D used to calculate the fourth difference 146 matching the third difference 144 is added is displayed in the first display region 36, and the pushing step frame 40C to which the third movement distance 112C used to calculate the third difference 144 is added is displayed in the second display region 38.

Therefore, with the endoscope apparatus 10 according to the second embodiment, in a case where the pushing step 41C precedes the pulling step 41D (see FIG. 13), and the controller 82C determines that the level of the identity between the lesion 42 shown in the pulling step frame 40D and the lesion 42 shown in the pushing step frame 40C is equal to or higher than the predetermined level, the doctor 12 can visually ascertain an aspect of the lesion 42 shown in the pulling step frame 40D displayed in the first display region 36 and an aspect of the lesion 42 shown in the pushing step frame 40C displayed in the second display region 38.

In addition, in the endoscope apparatus 10 according to the second embodiment, in a case where the pulling step 41D precedes the pushing step 41C (see FIG. 14), and the lesion 42 is shown in the pushing step frame 40C in the pushing step 41C, the controller 82C determines whether or not the third difference 144 and the fourth difference 146 match. The fourth difference 146 corresponds to a distance from the lesion 42 found in the pulling step 41D to the turning position P2B (see FIG. 14), and the third difference 144 corresponds to a distance from the turning position P2A to the lesion 42 found in the pushing step 41C. In a case where the third difference 144 and the fourth difference 146 match, the controller 82C determines that the level of the identity between the lesion 42 shown in the pushing step frame 40C and the lesion 42 shown in the pulling step frame 40D is equal to or higher than a predetermined level. In a case where the controller 82C determines that the level of the identity between the lesion 42 shown in the pushing step frame 40C and the lesion 42 shown in the pulling step frame 40D is equal to or higher than the predetermined level, the second display process 148 is executed by the controller 82C. In the second display process 148, the pushing step frame 40C to which the third movement distance 112C used to calculate the third difference 144 matching the fourth difference 146 is added is displayed in the first display region 36, and the pulling step frame 40D to which the fourth movement distance 112D used to calculate the fourth difference 146 is added is displayed in the second display region 38.

Therefore, with the endoscope apparatus 10 according to the second embodiment, in a case where the pulling step 41D precedes the pushing step 41C (see FIG. 14), and the controller 82C determines that the level of the identity between the lesion 42 shown in the pushing step frame 40C and the lesion 42 shown in the pulling step frame 40D is equal to or higher than the predetermined level, the doctor 12 can visually ascertain an aspect of the lesion 42 shown in the pushing step frame 40C displayed in the first display region 36 and an aspect of the lesion 42 shown in the pulling step frame 40D displayed in the second display region 38.

In addition, in the endoscope apparatus 10 according to the second embodiment, the pushing step tag 132 is added to the pushing step frame 40C, and the pulling step tag 134 is added to the pulling step frame 40D. The pushing step tag 132 is a tag for identifying that the pushing step frame 40C is the frame 40 obtained by imaging the region including the lesion 42 by the camera 52 in the pushing step 41C. The pulling step tag 134 is a tag for identifying that the pulling step frame 40D is the frame 40 obtained by imaging the region including the lesion 42 by the camera 52 in the pulling step 41D.

Therefore, with the endoscope apparatus 10 according to the second embodiment, it is possible to easily identify that the pushing step frame 40C is the frame 40 obtained by imaging the region including the lesion 42 by the camera 52 in the pushing step 41C, and the pulling step frame 40D is the frame 40 obtained by imaging the region including the lesion 42 by the camera 52 in the pulling step 41D. As a result, it is easy to perform a process using the pushing step frame 40C and the pulling step frame 40D.

In the first medical support process described in the second embodiment, a form example has been described in which the pushing step frame 40C is displayed in the second display region 38 on a condition that the third difference 144 and the fourth difference 146 match, but the present disclosure is not limited to this. For example, in a case where the pushing step 41C precedes the pulling step 41D (see FIG. 13), the pushing step frame 40C may be displayed in the second display region 38 on a condition that the pushing step frame 40C to which the third movement distance 112C used to calculate the third difference 144 matching the fourth difference 146 is added is continuously obtained for a second predetermined number of frames (for example, a plurality of frames designated in advance) or more. In this case, the pushing step frame 40C displayed in the second display region 38 may be, among a plurality of the pushing step frames 40C continuously obtained for the second predetermined number of frames or more, the pushing step frame 40C obtained first in time, may be the pushing step frame 40C obtained last in time, or may be the pushing step frame 40C obtained between the pushing step frame 40C obtained first in time and the pushing step frame 40C obtained last in time. In addition, among the plurality of pushing step frames 40C continuously obtained for the second predetermined number of frames or more, the pushing step frame 40C in which a position at which the lesion 42 is shown is closest to the center of the angle of view of the camera 52 may be displayed in the second display region 38. In addition, all the plurality of pushing step frames 40C continuously obtained for the second predetermined number of frames or more or a plurality of the pushing step frames 40C selected according to some rule may be displayed in the second display region 38 in a comparable state.

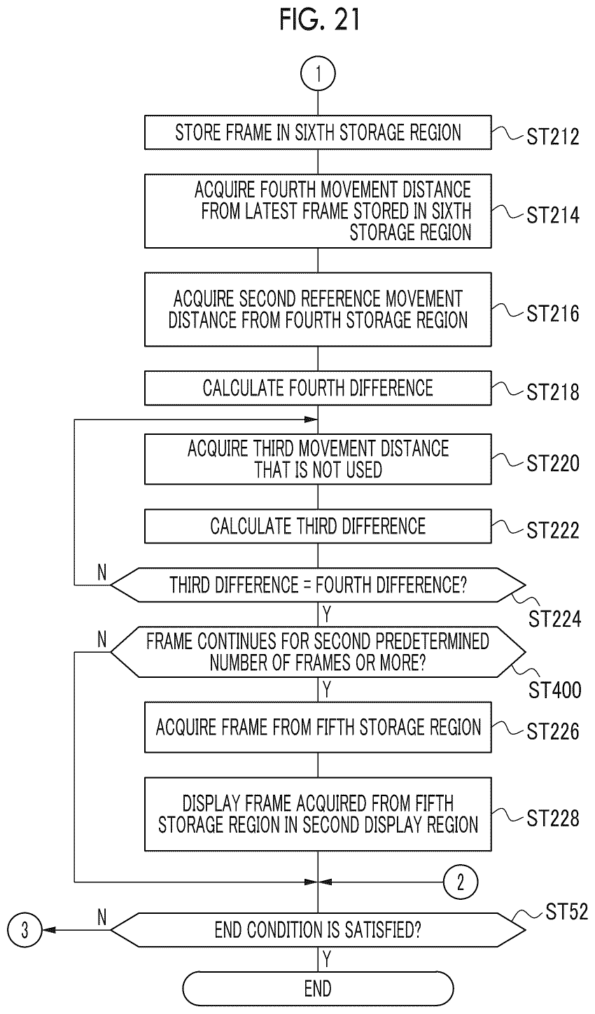

FIG. 21 shows an example of a flow of a first medical support process for realizing this form example. The first medical support process shown in FIG. 21 is different from the first medical support process shown in FIG. 19B in that a process of step ST400 is provided between the process of step ST224 and the process of step ST226. In step ST400 shown in FIG. 21, the controller 82C determines whether or not the pushing step frame 40C to which the third movement distance 112C used to calculate the third difference 144 matching the fourth difference 146 is added continues for the second predetermined of frames or more. In step ST400, in a case where the pushing step frame 40C to which the third movement distance 112C used to calculate the third difference 144 matching the fourth difference 146 is added does not continue for the second predetermined number of frames or more, a negative determination is made, and the first medical support process proceeds to step ST52. In step ST400, in a case where the pushing step frame 40C to which the third movement distance 112C used to calculate the third difference 144 matching the fourth difference 146 is added continues for the second predetermined number of frames or more, a positive determination is made, and the first medical support process proceeds to step ST226.

In this way, in a case where the pushing step 41C precedes the pulling step 41D (see FIG. 13), the pushing step frame 40C is displayed in the second display region 38 on a condition that the pushing step frame 40C to which the third movement distance 112C used to calculate the third difference 144 matching the fourth difference 146 is added is continuously obtained for the second predetermined number of frames or more, so that the doctor 12 can observe the same lesion 42 as the lesion 42 (in other words, the lesion 42 recognized by the recognition unit 82A) that is highly likely to be observed in the pushing step 41C by the doctor 12 in the pulling step 41D.

In addition, in a case where the pushing step 41D precedes the pushing step 41C (see FIG. 14), the pulling step frame 40D may be displayed in the second display region 38 on a condition that the pulling step frame 40D to which the fourth movement distance 112D used to calculate the fourth difference 146 matching the third difference 144 is added is continuously obtained for a third predetermined number of frames (for example, a plurality of frames designated in advance) or more. In this case, the pulling step frame 40D displayed in the second display region 38 may be, among a plurality of the pulling step frames 40D continuously obtained for the third predetermined number of frames or more, the pulling step frame 40D obtained first in time, may be the pulling step frame 40D obtained last in time, or may be the pulling step frame 40D obtained between the pulling step frame 40D obtained first in time and the pulling step frame 40D obtained last in time. In addition, among the plurality of pulling step frames 40D continuously obtained for the third predetermined number of frames or more, the pulling step frame 40D in which a position at which the lesion 42 is shown is closest to the center of the angle of view of the camera 52 may be displayed in the second display region 38. In addition, all the plurality of pulling step frames 40D continuously obtained for the third predetermined number of frames or more or a plurality of the pulling step frames 40D selected according to some rule may be displayed in the second display region 38 in a comparable state.

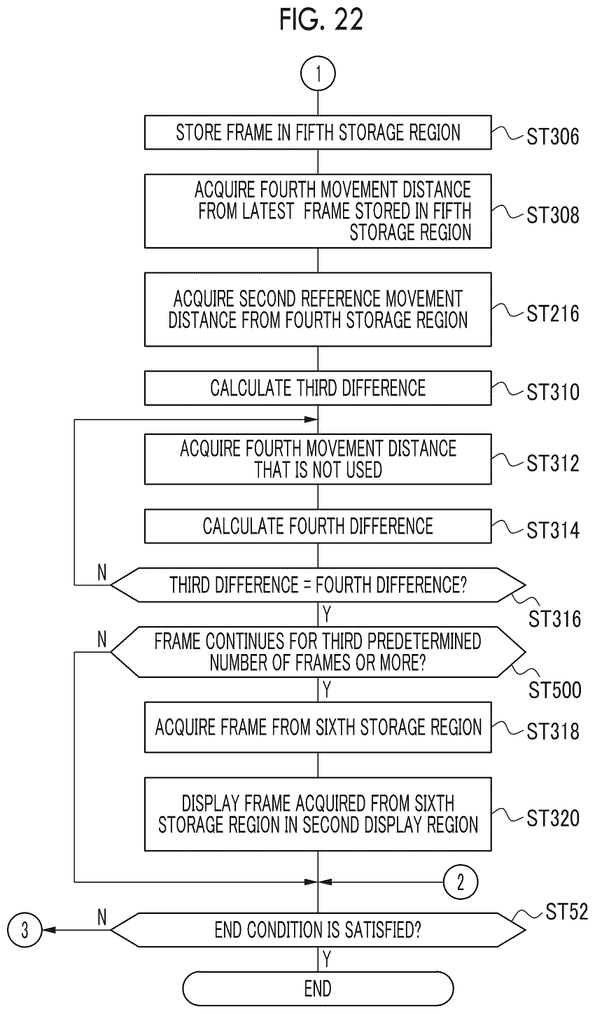

FIG. 22 shows an example of a flow of a second medical support process for realizing this form example. The second medical support process shown in FIG. 22 is different from the second medical support process shown in FIG. 20B in that a process of step ST500 is provided between the process of step ST316 and the process of step ST318. In step ST500 shown in FIG. 22, the controller 82C determines whether or not the pulling step frame 40D to which the fourth movement distance 112D used to calculate the fourth difference 146 matching the third difference 144 is added continues for the third predetermined number of frames or more. In step ST500, in a case where the pulling step frame 40D to which the fourth movement distance 112D used to calculate the fourth difference 146 matching the third difference 144 is added does not continue for the third predetermined number of frames or more, a negative determination is made, and the second medical support process proceeds to step ST52. In step ST500, in a case where the pulling step frames 40D to which the fourth movement distance 112D used to calculate the fourth difference 146 matching the third difference 144 is added continues for the third predetermined number of frames or more, a positive determination is made, and the second medical support process proceeds to step ST318.

In this way, in a case where the pulling step 41D precedes the pushing step 41C (see FIG. 14), the pulling step frame 40D is displayed in the second display region 38 on a condition that the pulling step frames 40D to which the fourth movement distance 112D used to calculate the fourth difference 146 matching the third difference 144 is added is continuously obtained for the third predetermined number of frames or more, so that the doctor 12 can observe the same lesion 42 as the lesion 42 (in other words, the lesion 42 recognized by the recognition unit 82A) that is highly likely to be observed in the pulling step 41D by the doctor 12 in the pushing step 41C.

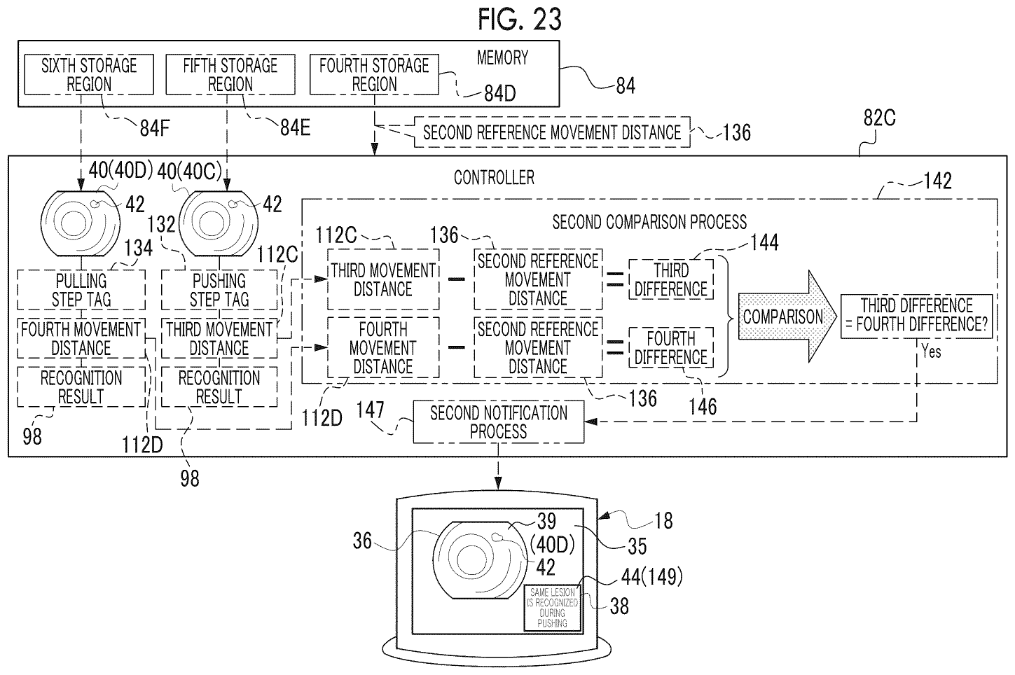

In the second embodiment, although a form example has been described in which the controller 82C executes the second display process 148, this is merely an example. For example, as shown in FIG. 23, the controller 82C may execute a second notification process 147. The second notification process 147 is a process of issuing a notification that the level of the identity between the lesion 42 shown in the pushing step frame 40C and the lesion 42 shown in the pulling step frame 40D is equal to or higher than a predetermined level in a case where the level of the identity between the lesion 42 shown in the pushing step frame 40C and the lesion 42 shown in the pulling step frame 40D is equal to or higher than the predetermined level.

Examples of the notification include an example in which a message 149 for notifying that the level of the identity between the lesion 42 shown in the pushing step frame 40C and the lesion 42 shown in the pulling step frame 40D is equal to or higher than the predetermined level is displayed in the second display region 38 as one of pieces of the assistance information 44. In addition, a mark, a numerical value, or the like for notifying that the level of the identity between the lesion 42 shown in the pushing step frame 40C and the lesion 42 shown in the pulling step frame 40D is equal to or higher than the predetermined level may be displayed in the second display region 38 as one of pieces of the assistance information 44. In addition, the pushing step frame 40C or the pulling step frame 40D may also be displayed in the second display region 38 as in the second embodiment. In addition, information for giving a notification that the level of the identity between the lesion 42 shown in the pushing step frame 40C and the lesion 42 shown in the pulling step frame 40D is equal to or higher than the predetermined level may be displayed in a region other than the second display region 38 in the screen 35 or may be displayed on a screen of a display device different from the display device 18. In addition, a voice for notifying that the level of the identity between the lesion 42 shown in the pushing step frame 40C and the lesion 42 shown in the pulling step frame 40D is equal to or higher than the predetermined level may be output from a speaker (not shown).

In this way, by executing the second notification process 147 by the controller 82C, the doctor 12 can perceive that the level of the identity between the lesion 42 shown in the pushing step frame 40C and the lesion 42 shown in the pulling step frame 40D is equal to or higher than the predetermined level.

In the example shown in FIG. 23, as an example of the notification in a case where the pushing step 41C precedes the pulling step 41D (see FIG. 13), an example of an aspect is shown in which the pulling step frame 40D is displayed in the first display region 36 and a message indicating that the same lesion 42 as the lesion 42 shown in the pulling step frame 40D displayed in the first display region 36 is also recognized in the pushing step 41C (in the example shown in FIG. 23, a message “same lesion is recognized during pushing”) is displayed in the second display region 38 as the message 149, but this is merely an example. In a case where the pulling step 41D precedes the pushing step 41C (see FIG. 14), the pushing step frame 40C may be displayed in the first display region 36, and a message indicating that the same lesion 42 as the lesion 42 shown in the pushing step frame 40C displayed in the first display region 36 is also recognized in the pulling step 41D may be displayed in the second display region 38.

### Other Embodiments

In each of the embodiments described above, a form example has been described in which the estimation unit 82B estimates the movement distance 112 by executing the movement estimation process 110. However, in a case where the estimation unit 82B cannot estimate the movement distance 112 by executing the movement estimation process 110, the estimation unit 82B may estimate the movement distance 112 based on the movement distance 112 estimated in the past and an elapsed time from switching from a first tag to a second tag as an exceptional process. In this case, for example, the estimation unit 82B assumes that a movement speed of the camera 52 is constant and estimates the movement distance 112 using a linear interpolation, a linear Kalman filter, or the like.

Here, a first example of the first tag includes the insertion step tag 116. A first example of the second tag in a case where the first tag is the insertion step tag 116 includes the removal step tag 118. Further, a second example of the first tag includes the pushing step tag 132. A second example of the second tag in a case where the first tag is the pushing step tag 132 includes the pulling step tag 134. A third example of the first tag includes the pulling step tag 134. A third example of the second tag in a case where the first tag is the pulling step tag 134 includes the pushing step tag 132.

In each of the embodiments described above, although a form example has been described in which the frame 40 is displayed in the first display region 36 and the second display region 38, the lesion position map 104 may be displayed in at least one of the first display region 36 or the second display region 38. For example, the lesion position map 104 corresponding to the frame 40 displayed in the first display region 36 may be displayed in the first display region 36 together with the frame 40 displayed in the first display region 36 or instead of the frame 40 displayed in the first display region 36, or the lesion position map 104 corresponding to the frame 40 displayed in the second display region 38 may be displayed in the second display region 38 together with the frame 40 displayed in the second display region 38 or instead of the frame 40 displayed in the second display region 38.

In each of the embodiments described above, although a form example has been described in which one frame 40 is displayed in the second display region 38, this is merely an example, and a plurality of the frames 40 may be displayed in the second display region 38. The plurality of frames 40 may be displayed side by side in a comparable state, or may be displayed in a descending order of a similarity with the lesion 42 shown in the frame 40 displayed in the first display region 36. Examples of the plurality of frames 40 include a plurality of frames 40 continuously obtained. In addition, one or more frames 40 showing the lesion 42 of which a similarity with the lesion 42 shown in the frame 40 displayed in the first display region 36 is equal to or higher than a certain level may be displayed in the second display region 38.

In each of the embodiments described above, although a form example has been described in which the frame 40 is displayed in the second display region 38, the entire frame 40 may not be displayed in the second display region 38, and a part of an image region (for example, a lesion region indicating the lesion 42) included in the frame 40 may be displayed in the second display region 38.

In each of the embodiments described above, although a form example has been described in which the first specifying process is executed as a process for specifying that the lesion 42 shown in the insertion step frame 40A and the lesion 42 shown in the removal step frame 40B are the same, the present disclosure is not limited to this. For example, in a case where the lesion 42 shown in the insertion step frame 40A and the lesion 42 shown in the removal step frame 40B are not the same, a process for specifying that the lesion 42 shown in the insertion step frame 40A and the lesion 42 shown in the removal step frame 40B are not the same (for example, a display process and/or a notification process performed in the same manner as in the first display process and the first notification process described above) may be executed. In this way, the process for specifying that the lesion 42 shown in the insertion step frame 40A and the lesion 42 shown in the removal step frame 40B are not the same, so that the doctor 12 can ascertain that the lesion 42 found in the insertion step 41A is not imaged in the removal step 41B.

In addition, in the same manner as described above, in a case where the lesion 42 shown in the pushing step frame 40C and the lesion 42 shown in the pulling step frame 40D are not the same, a process for specifying that the lesion 42 shown in the pushing step frame 40C and the lesion 42 shown in the pulling step frame 40D are not the same (for example, a display process and/or a notification process performed in the same manner as in the second display process and the second notification process described above) may be executed.

In each of the embodiments described above, a form example has been described in which the position of the camera 52 inserted into the large intestine 28 inside the large intestine 28 is specified based on the movement distance 112, but the present disclosure is not limited to this. For example, the position of the camera 52 inside the large intestine 28 may be specified based on a detection result by an endoscope insertion shape observation device (commonly known as a colonoscope navigation).

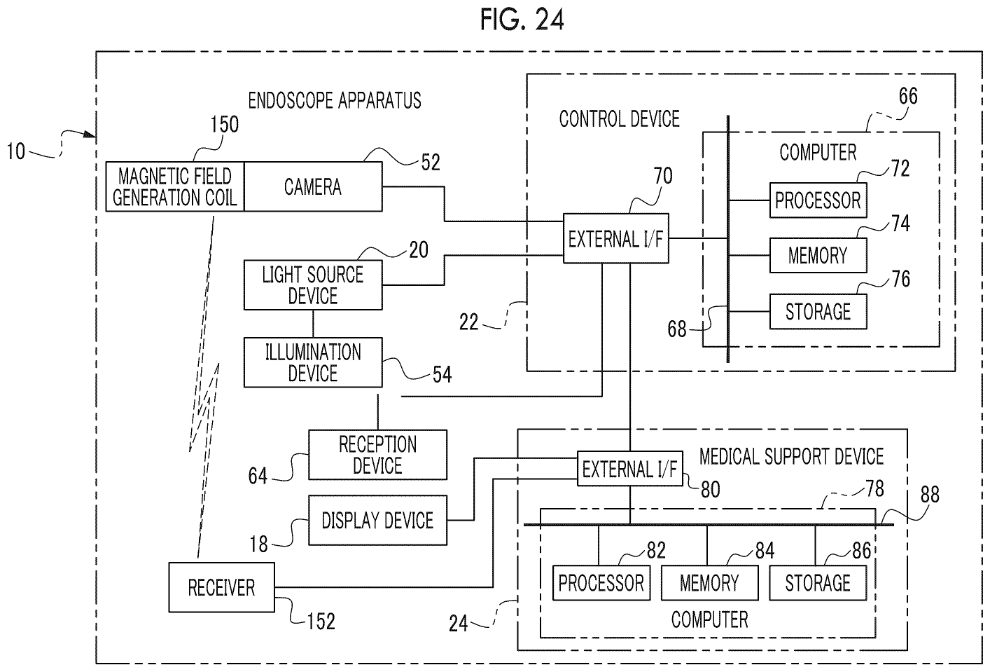

In this case, as shown in FIG. 24 as an example, the camera 52 is provided with a magnetic field generation coil 150, and a receiver 152 is connected to the external I/F 80. A magnetic field generated from the magnetic field generation coil 150 is received by the receiver 152. The processor 82 detects a behavior (for example, a position and an orientation) of the camera 52 inside the large intestine 28 based on a reception result by the receiver 152, and specifies the position of the camera 52 inside the large intestine 28 based on a detection result. In this way, the behavior of the camera 52 inside the large intestine 28 is accurately detected, so that it is possible to accurately specify the position of the camera 52 inside the large intestine 28.

The endoscope insertion shape observation device described here is merely an example, and a sensor other than the endoscope insertion shape observation device may be used. For example, the position of the camera 52 inside the large intestine 28 may be specified based on a detection result of a sensor capable of detecting the behavior of the camera 52 inside the large intestine 28, such as an acceleration sensor, a gyro sensor, and/or a magnetic sensor.

In each of the embodiments described above, the lesion 42 is described as an example of a “first feature region”, a “second feature region”, a “third feature region”, and a “fourth feature region” according to the present disclosure, but the present disclosure is not limited to this. The medical support process described above is established even in a case where a resection region, a bleeding region, a marking region, an organ, a treatment tool (for example, a hemostatic clip placed in the body), or the like is applied instead of the lesion 42.

In each of the embodiments described above, although a form example has been described in which the frame 40 is input to the recognition unit 82A and the processing result by the medical support process is output from the controller 82C, the present disclosure is not limited to this. For example, instruction data (so-called prompt) including the frame 40 may be input to so-called generation AI, and the processing result by the medical support process may be output from the generation AI. Examples of the generation AI include ChatGPT using GPT-4 (Internet search <https://openai.com/gpt-4>).

In addition, instruction data including at least a part of the processing result by the medical support process (for example, the frame 40 displayed on the screen 35 by executing the first display process, the frame 40 displayed on the screen 35 by executing the second display process, information notified by executing the first notification process, and/or information notified by executing the second notification process) may be used as input information for the generation AI. Examples of the information output from the generation AI include information related to an operation content of the camera 52, information indicating a content of a medical treatment that is recommended to be performed during the endoscopy, and/or information indicating a content of a medical treatment that is recommended to be performed after the endoscopy. The information output from the generation AI may be stored in various storage regions (for example, the storage 76 and/or 86), displayed on the display device 18 as the assistance information 44, printed on a medium by the printer, or output from a speaker as a voice.

In each of the embodiments described above, the recognition process 96 using AI in a bounding box method has been illustrated, but this is merely an example. For example, a recognition process using AI in a segmentation method may be performed instead of the recognition process 96 using AI in a bounding box method. In addition, a recognition process in a non-AI method (for example, a template matching method) may be performed instead of the recognition process in an AI method, or a recognition process in which the non-AI method and the AI method are combined may be performed.

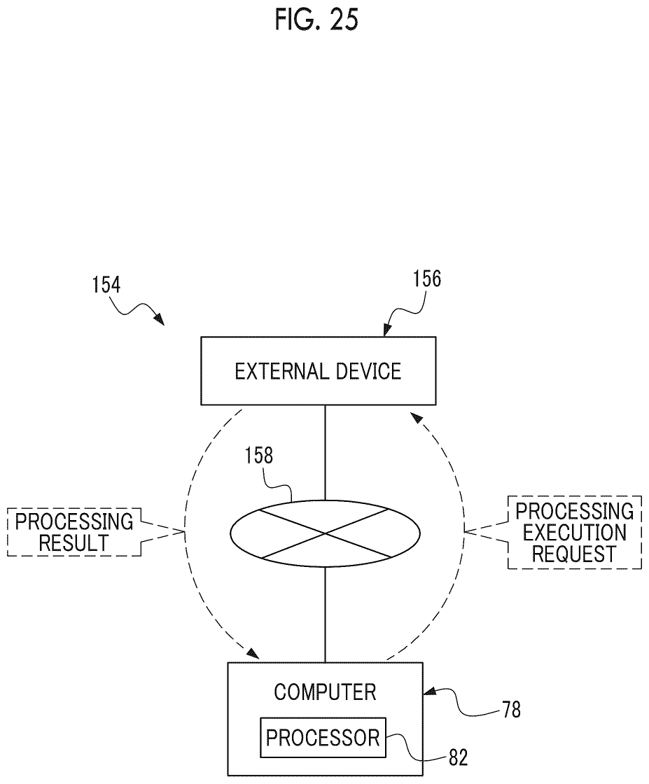

In each of the embodiments described above, a form example has been described in which the medical support process is performed by the computer 78, but the present disclosure is not limited to this. At least some of processing included in the medical support process may be performed by a device provided outside the computer 78. Hereinafter, an example of this case will be described with reference to FIG. 25.

FIG. 25 is a conceptual diagram showing an example of a configuration of an endoscope apparatus 154. The endoscope apparatus 154 is an example of an “endoscope apparatus” according to the present disclosure. The endoscope apparatus 154 is different from the endoscope apparatus 10 described in each of the embodiments described above in that an external device 156 is provided.

The external device 156 is communicably connected to the computer 78 via a network 158 (for example, a WAN and/or a LAN).

Examples of the external device 156 include at least one server that directly or indirectly performs transmission and reception of data with the computer 78 via the network 158. The external device 156 receives a processing execution instruction given from the processor 82 of the computer 78 via the network 158. Then, the external device 156 executes processing according to the received processing execution instruction and transmits a processing result to the computer 78 via the network 158. In the computer 78, the processor 82 receives the processing result transmitted from the external device 156 via the network 158 and executes a process using the received processing result.

Examples of the processing execution instruction include an instruction for the external device 156 to execute at least a part of the medical support process. First examples of at least a part (that is, processing executed by the external device 156) of the medical support process include the recognition process 96. In this case, the external device 156 executes the recognition process 96 in response to the processing execution instruction given from the processor 82 via the network 158 and transmits the recognition result 98 to the computer 78 via the network 158. In the computer 78, the processor 82 receives the recognition result 98 and executes the same processing as in each of the embodiments described above by using the received recognition result 98.

Second examples of at least a part of the medical support process (that is, processing executed by the external device 156) include processing by the controller 82C. In this case, the external device 156 executes the processing by the controller 82C in response to the processing execution instruction given from the processor 82 via the network 158, and transmits the processing result (for example, the frame 40 displayed on the screen 35 by executing the first display process, the frame 40 displayed on the screen 35 by executing the second display process, the information notified by executing the first notification process, and/or the information notified by executing the second notification process) to the computer 78 via the network 158. In the computer 78, the processor 82 receives the processing result and executes the same processing as in each of the embodiments described above (for example, the display using the display device 18) by using the received processing result.

For example, the external device 156 is realized by cloud computing. It should be noted that the cloud computing is merely an example, and the external device 156 may be realized by network computing such as fog computing, edge computing, or grid computing. Instead of the server, at least one personal computer or the like may be used as the external device 156. In addition, a computing device having a communication function equipped with a plurality of types of AI functions may be used.

In each of the embodiments described above, a form example has been described in which the medical support program 90 is stored in the storage 86, but the present disclosure is not limited to this. For example, the medical support program 90 may be stored in a portable computer-readable non-transitory storage medium, such as an SSD or a USB memory. The medical support program 90 stored in the non-transitory storage medium is installed in the computer 78 of the endoscope apparatus 10. The processor 82 executes the medical support process according to the medical support program 90.

In addition, the medical support program 90 may be stored in a storage device of another computer, server, or the like connected to the endoscope apparatus 10 via a network, and the medical support program 90 may be downloaded and installed in the computer 78 in response to a request from the endoscope apparatus 10.

It is not necessary to store all the medical support programs 90 in a storage device of another computer, server device, or the like connected to the endoscope apparatus 10 or to store all the medical support programs 90 in the storage 86, and a part of the medical support programs 90 may be stored.

The following various processors can be used as hardware resources for executing the medical support process. Examples of the processor include a CPU which is a general-purpose processor that executes software, that is, a program, to function as the hardware resource executing the medical support process. In addition, examples of the processor include a dedicated electric circuit which is a processor having a circuit configuration specially designed for executing specific processing, such as an FPGA, a PLD, or an ASIC. A memory is incorporated in or connected to any processor, and any processor executes the medical support process using the memory.

The hardware resource for executing the medical support process may be configured by one of the various processors or by a combination of two or more processors of the same type or different types (for example, a combination of a plurality of FPGAs or a combination of a CPU and an FPGA). Further, the hardware resource for executing the medical support process may be one processor.

As an example of the configuration using one processor, first, there is a form in which one processor is configured by a combination of one or more CPUs and software, and the processor functions as the hardware resource for executing the medical support process. Second, as typified by a SoC or the like, there is a form in which a processor that realizes all functions of a system including a plurality of hardware resources executing the medical support process with one IC chip is used. As described above, the medical support process is realized using one or more of the various processors as the hardware resource.

Further, as a hardware structure of these various processors, more specifically, an electrical circuit in which circuit elements such as semiconductor elements are combined can be used. Further, the above-described medical support process is only an example. Therefore, it is needless to say that unnecessary steps may be deleted, new steps may be added, or a processing order may be changed without departing from the gist of the present disclosure.

The above-described contents and illustrated contents are detailed descriptions of parts related to the present disclosure, and are merely examples of the present disclosure. For example, the above descriptions related to configurations, functions, operations, and advantageous effects are descriptions related to examples of configurations, functions, operations, and advantageous effects of the parts related to the present disclosure. Therefore, it is needless to say that unnecessary parts may be deleted, or new elements may be added or replaced with respect to the above-described contents and illustrated contents without departing from the gist of the present disclosure. In order to avoid complications and easily understand the parts according to the present disclosure, in the above-described contents and illustrated contents, common technical knowledge and the like that do not need to be described to implement the present disclosure are not described.

All documents, patent applications, and technical standards described in the present specification are incorporated in the present specification by reference to the same extent as in a case where each document, patent application, and technical standard are specifically and individually noted to be incorporated by reference.

What is claimed is:

1. A medical support device comprising a processor,
wherein the processor is configured to:
acquire position specifying information for specifying a position of a camera inserted into a body inside the body; and
execute, based on insertion position specifying information which is the position specifying information corresponding to an insertion step frame obtained by imaging a first feature region in the body by the camera in an insertion step of inserting the camera into the body, and removal position specifying information which is the position specifying information corresponding to a removal step frame obtained by imaging a second feature region in the body by the camera in a removal step of removing the camera from the body, a first specifying process of specifying an identity between the first feature region and the second feature region.

2. The medical support device according to claim 1,
wherein the first specifying process includes a first notification process of issuing a notification that a level of the identity between the first feature region and the second feature region is equal to or higher than a first predetermined level in a case where the level of the identity between the first feature region and the second feature region is equal to or higher than the first predetermined level.

3. The medical support device according to claim 1,
wherein the first specifying process includes a first display process of displaying a first image for specifying at least the first feature region shown in the insertion step frame and a second image for specifying at least the second feature region shown in the removal step frame on a first screen in a comparable state, in a case where a level of the identity between the first feature region and the second feature region is equal to or higher than a second predetermined level.

4. The medical support device according to claim 1,
wherein the processor is configured to:
acquire the insertion step frame; and
execute the first specifying process on a condition that a plurality of the insertion step frames, which is equal to or more than a first predetermined number of frames, are continuously acquired.

5. The medical support device according to claim 1,
wherein an insertion step identifier for identifying that the insertion step frame is a frame obtained by imaging the first feature region by the camera in the insertion step is added to the insertion step frame, and
a removal step identifier for identifying that the removal step frame is a frame obtained by imaging the second feature region by the camera in the removal step is added to the removal step frame.

6. The medical support device according to claim 1,
wherein the processor is configured to execute based on pushing position specifying information which is the position specifying information corresponding to a pushing step frame obtained by imaging a third feature region in the body by the camera in a pushing step in which the camera is pushed into a far side inside the body, and pulling position specifying information which is the position specifying information corresponding to a pulling step frame obtained by imaging a fourth feature region in the body by the camera in a pulling step in which the camera is pulled from an inside of the body to a near side, a second specifying process of specifying an identity between the third feature region and the fourth feature region.

7. A medical support device comprising a processor,
wherein the processor is configured to:
acquire position specifying information for specifying a position of a camera inserted into a body inside the body; and
execute, based on pushing position specifying information which is the position specifying information corresponding to a pushing step frame obtained by imaging a third feature region in the body by the camera in a pushing step in which the camera is pushed into a far side inside the body, and pulling position specifying information which is the position specifying information corresponding to a pulling step frame obtained by imaging a fourth feature region in the body by the camera in a pulling step in which the camera is pulled from the far side to a near side, a second specifying process of specifying an identity between the third feature region and the fourth feature region.

8. The medical support device according to claim 6,
wherein the second specifying process includes a second notification process of issuing a notification that a level of the identity between the third feature region and the fourth feature region is equal to or higher than a third predetermined level in a case where the level of the identity between the third feature region and the fourth feature region is equal to or higher than the third predetermined level.

9. The medical support device according to claim 6,
wherein the second specifying process includes a second display process of displaying a third image for specifying at least the third feature region shown in the pushing step frame and a fourth image for specifying at least the fourth feature region shown in the pulling step frame on a second screen in a comparable state, in a case where a level of the identity between the third feature region and the fourth feature region is equal to or higher than a fourth predetermined level.

10. The medical support device according to claim 6,
wherein the processor is configured to:
acquire the pushing step frame; and
execute the second specifying process on a condition that a plurality of the pushing step frames, which is equal to or more than a second predetermined number of frames, are continuously acquired in a case where the pushing step precedes the pulling step.

11. The medical support device according to claim 6,
wherein the processor is configured to:
acquire the pulling step frame; and
execute the second specifying process on a condition that a plurality of the pulling step frames, which is equal to or more than a third predetermined number of frames, are continuously acquired in a case where the pulling step precedes the pushing step.

12. The medical support device according to claim 6,
wherein a pushing step identifier for identifying that the pushing step frame is a frame obtained by imaging the third feature region by the camera in the pushing step is added to the pushing step frame, and
a pulling step identifier for identifying that the pulling step frame is a frame obtained by imaging the fourth feature region by the camera in the pulling step is added to the pulling step frame.

13. The medical support device according to claim 6,
wherein the third feature region and the fourth feature region are lesions.

14. The medical support device according to claim 1,
wherein the first feature region and the second feature region are lesions.

15. The medical support device according to claim 1,
wherein the position is estimated based on a plurality of frames obtained by imaging an inside of the body by the camera.

16. The medical support device according to claim 1,
wherein the position specifying information is defined based on a movement distance of the camera inside the body.

17. An endoscope apparatus comprising:
the medical support device according to claim 1; and
the camera.

18. A medical support method comprising:
acquiring position specifying information for specifying a position of a camera inserted into a body inside the body; and
executing, based on insertion position specifying information which is the position specifying information corresponding to an insertion step frame obtained by imaging a first feature region in the body by the camera in an insertion step of inserting the camera into the body, and removal position specifying information which is the position specifying information corresponding to a removal step frame obtained by imaging a second feature region in the body by the camera in a removal step of removing the camera from the body, a first specifying process of specifying an identity between the first feature region and the second feature region.

19. A medical support method comprising:
acquiring position specifying information for specifying a position of a camera inserted into a body inside the body; and
executing, based on pushing position specifying information which is the position specifying information corresponding to a pushing step frame obtained by imaging a third feature region in the body by the camera in a pushing step in which the camera is pushed into a far side inside the body, and pulling position specifying information which is the position specifying information corresponding to a pulling step frame obtained by imaging a fourth feature region in the body by the camera in a pulling step in which the camera is pulled from the far side to a near side, a second specifying process of specifying an identity between the third feature region and the fourth feature region.

20. A non-transitory computer-readable storage medium storing a program executable by a computer to execute a process comprising:
acquiring position specifying information for specifying a position of a camera inserted into a body inside the body; and
executing, based on insertion position specifying information which is the position specifying information corresponding to an insertion step frame obtained by imaging a first feature region in the body by the camera in an insertion step of inserting the camera into the body, and removal position specifying information which is the position specifying information corresponding to a removal step frame obtained by imaging a second feature region in the body by the camera in a removal step of removing the camera from the body, a first specifying process of specifying an identity between the first feature region and the second feature region.

21. A non-transitory computer-readable storage medium storing a program executable by a computer to execute a process comprising:
acquiring position specifying information for specifying a position of a camera inserted into a body inside the body; and
executing, based on pushing position specifying information which is the position specifying information corresponding to a pushing step frame obtained by imaging a third feature region in the body by the camera in a pushing step in which the camera is pushed into a far side inside the body, and pulling position specifying information which is the position specifying information corresponding to a pulling step frame obtained by imaging a fourth feature region in the body by the camera in a pulling step in which the camera is pulled from the far side to a near side, a second specifying process of specifying an identity between the third feature region and the fourth feature region.
